(19) United States
(12) Patent Application Publication
(10) Pub. No.: US 2026/0124157 A1
(43) Pub. Date: May 7, 2026

(54) METHODS OF SELF-AMPLIFYING RNA-LIPID NANOPARTICLE MANUFACTURE AND COMPOSITIONS DERIVED THEREFROM

(71) Applicant: Genvax Technologies, Inc., Ames, IA (US)

(72) Inventors: Robert HARRIS, Ames, IA (US); Lance DAHARSH, Ames, IA (US)

(21) Appl. No.: 19/294,990

(22) Filed: Aug. 8, 2025

Related U.S. Application Data
(60) Provisional application No. 63/681,717, filed on Aug. 9, 2024.

(51) Int. Cl. A61K 9/51; A61K 31/7105; A61K 31/711; A61K 47/22; A61K 47/24; A61K 47/28

(52) U.S. Cl. CPC A61K 9/5192; A61K 9/5123; A61K 9/5146; A61K 31/7105; A61K 31/711; A61K 47/22; A61K 47/24; A61K 47/28

(57) ABSTRACT

Disclosed herein are methods of increasing the potency of saRNA encapsulated lipid nanoparticles (LNPs) through a novel and surprisingly superior LNP manufacturing technique. The method disclosed herein overcomes technical difficulties and high costs associated with previous LNP manufacturing techniques. The methods disclosed herein, therefore, greatly improve the industrial production of LNPs in unexpected ways thereby providing more potent and less expensive LNPs for nucleic acid delivery.

### CROSS REFERENCE TO RELATED APPLICATIONS

This application claims the benefit of U.S. provisional patent application No. 63/681,717, which was filed on Aug. 9, 2024, and is incorporated herein by reference in its entirety.

### FIELD OF THE INVENTION

The invention is in the field of nanoparticle manufacturing for the delivery of self-amplifying RNA. Accordingly, the invention encompasses a simplified and economical method for rapidly preparing self-amplifying RNA-encapsulated lipid nanoparticles with greater potency and storage stability.

### BACKGROUND OF THE INVENTION

The delivery of biologically active agents (including therapeutically relevant compounds) to subjects is often hindered by difficulties in the compounds reaching the target cell or tissue. In particular, the trafficking of many biologically active agents into living cells is highly restricted by the complex membrane systems of the cells. These restrictions can result in the need to use much higher concentrations of biologically active agents than is desirable to achieve a result, which increases the risk of toxic effects and side effects. One solution to this problem is to utilize specific carrier molecules and carrier compositions, which allow selective entry into the cell. Lipid carriers, biodegradable polymers, and various conjugate systems can be used to improve the delivery of biologically active agents to cells.

One class of biologically active agents that is particularly difficult to deliver to cells is a bio-therapeutic (including peptides, proteins, nucleosides, nucleotides, polynucleotides, nucleic acids and derivatives, such as mRNA, RNAi/siRNA, and self-replicating/amplifying RNA (i.e., saRNA). In general, nucleic acids are stable for only a limited duration in cells or bodily fluids. The development of CRISPR/CAS9, RNA interference, RNAi therapy, mRNA therapy, RNA drugs, antisense therapy, gene therapy, and nucleic acid vaccines (e.g., saRNA vaccines), among others, has increased the need for an effective means of introducing active nucleic acid agents into cells. For these reasons, compositions that can stabilize and deliver nucleic acid-based agents into cells are of interest.

An alternative approach is to use delivery compositions incorporating ionizable or nonionizable cationic lipids, which interact with a biologically active agent at one part and interact with a membrane system at another part. Such compositions are reported to provide liposomes, micelles, lipoplexes, or lipid nanoparticles, depending on the composition and method of preparation (for reviews, see Felgner, 1990, Advanced Drug Delivery Reviews, 5, 162-187; Felgner, 1993, J. Liposome Res., 3, 3-16; Gallas, 2013, Chem. Soc. Rev., 42, 7983-7997; Falsini, 2013, J. Med. Chem. dx.doi.org/10.1021/jm400791q; and references therein).

Lipid formulations are attractive carriers since they can protect biological molecules from degradation while improving their cellular uptake. Out of the various classes of lipid formulations, formulations that contain ionizable or non-ionizable cationic lipids are commonly used for delivering polyanions (e.g., nucleic acids). Such formulations can be formed using cationic lipids alone and optionally including other lipids and amphiphiles such as cholesterol and phosphatidylcholine. It is well known in the art that both the composition of the lipid formulation as well as its method of preparation affect the structure and size of the resultant nanoparticle or aggregate (Leung, 2012, J. Phys Chem. C, 116, 18440-18450).

A variety of methods have been developed to formulate LNP systems containing genetic drugs. These methods include mixing lipid dissolved in ethanol with an aqueous media containing nucleic acids to produce LNPs with 150 nm or less diameters. These methods rely on the presence of ionizable or non-ionizable cationic lipids to achieve encapsulation of oligonucleotide (OGN) and poly(ethylene glycol) (PEG) to inhibit aggregation and the formation of large structures. The properties of the LNP systems produced, including size and OGN encapsulation efficiency, are sensitive to a variety of formulation parameters such as ionic strength, lipid and ethanol concentration, pH, nucleic acid concentration, and mixing rates. In general, parameters such as the relative lipid and nucleic acid concentrations at the time of mixing, as well as the mixing rates are difficult to control using current formulation procedures, resulting in variability in the characteristics of the LNP produced, both within and between preparations.

The use of concentrations of ethanol and removal by dialysis in encapsulating RNA, particularly self-amplifying RNA (i.e., saRNA), suffers from a number of limitations. The method is sensitive to minor changes in the ionic strength of the formulation buffer; changes as small as 10 mM result in a dramatic decrease in encapsulation efficiency. Even when LNPs are formed under ideal conditions, the dialysis method results in significant losses in the amount and integrity of saRNA. The dialysis method is also difficult to scale to the size required to support preclinical and clinical development. For this reason, the inventors sought to develop a more simple, robust, and fully scalable method for the preparation of saRNA LNPs.

Despite advances in the development of methods for LNP systems containing genetic drugs, a need exists for methods for preparing lipid nanoparticles containing therapeutic materials (e.g., mRNA or saRNA), as well as improved lipid nanoparticles containing therapeutic materials. The present invention seeks to fulfill this need and provides further related advantages.

### SUMMARY OF THE INVENTION

The invention encompasses a dialysis-free method for making a lipid nanoparticle (“LNP”) comprising a nucleic acid (e.g., DNA, RNA, mRNA, saRNA), preferably saRNA, providing an aqueous nucleic acid solution comprising of at least one nucleic acid at a nucleic acid concentration; providing an organic solvent lipid solution comprising at least one lipid at a lipid concentration; and combining a portion of the nucleic acid solution and a portion of the lipid solution to create a mixing solution with an organic solvent (e.g., ethanol) concentration, adjusting the pH in the mixing solution to physiological pH, and dilution with a volume of cryoprotectant buffer that reduces the organic solvent to a non-toxic concentration in the payload encapsulated LNPs. The method yields a greater recovery, integrity, potency, and storage stability of saRNA lipid nanoparticles, resulting in lower cost of the final product, than traditional methods that utilize dialysis to remove ethanol.

In certain embodiments, saRNA comprising LNPs formed by the methods of the invention have a greater potency than lipid nanoparticles prepared by dialysis.

In certain embodiments, saRNA comprising LNPs formed by the methods of the invention have a greater recovery than lipid nanoparticles prepared by dialysis.

In certain embodiments, saRNA comprising LNPs formed by the methods of the invention have a greater integrity than lipid nanoparticles prepared by dialysis.

In certain embodiments, saRNA comprising LNPs formed by the methods of the invention have a greater storage stability than lipid nanoparticles prepared by using dialysis.

In one embodiment, the portion of the nucleic acid solution and the portion of the lipid solution are combined in volume ratios selected from the group consisting of 1:1, 2:1, 3:1, 4:1, 5:1, 6:1 and 7:1. In another embodiment, the LNPs have an average a diameter in the range of about 40 to about 150 nanometers. In a further embodiment, the LNPs have an average diameter in the range of about 50 to about 100 nanometers. In still another embodiment, the LNPs have a nucleic acid encapsulation efficiency of about 40 to about 100%. In yet a further embodiment, the LNPs have a nucleic acid encapsulation efficiency of about 50% to about 99%. In another embodiment, the LNPs have a nucleic acid encapsulation efficiency of about 60% to about 85%. In still another embodiment, the LNPs have a nucleic acid encapsulation efficiency of about 70% to about 80%.

In a further embodiment, at least one nucleic acid is DNA or RNA. In still a further embodiment, at least one nucleic acid is RNA. In yet another embodiment, at least one nucleic acid is mRNA. In yet another embodiment, at least one nucleic acid is saRNA. In yet another embodiment, at least one nucleic acid is mRNA or saRNA encoding at least one open reading frame. In still another embodiment, at least one nucleic acid is mRNA or saRNA encoding at least one open reading frame encoding an immunogen.

In a further embodiment, the nucleic acid solution comprises a buffer. In yet another embodiment, the nucleic acid concentration is at least or about 0.2 to about 3 mg/ml. In a further embodiment, the nucleic acid concentration is at least or about 0.30 to about 3 mg/ml. In another embodiment, the nucleic acid concentration is at least or about 0.40 to about 3 mg/ml. In a further embodiment, the nucleic acid concentration is at least or about 0.50 to about 3 mg/ml. In still another embodiment, the nucleic acid concentration is at least or about 0.60 to about 3 mg/ml. In a further embodiment, the nucleic acid concentration is at least or about 0.70 to about 3 mg/ml. In still a further embodiment, the nucleic acid concentration is at least or about 1 to about 3 mg/ml.

In still another embodiment, the lipid solution comprises an organic solvent selected from the group consisting of ethanol, acetone, propanol, isopropanol, and THE or combinations thereof. In still another embodiment, at least one lipid in the lipid solution is a cationic lipid, for example, selected from the group consisting of MC3, KC2, DLin, DODMA, DODAP, and a combination thereof. In still another embodiment, at least one lipid in the lipid solution is a cationic lipid. In yet a further embodiment, at least one lipid in the lipid solution is an ionizable cationic lipid.

In yet a further embodiment, the final lipid concentration is at least or about 1 mM to about 50 mM. In yet another embodiment, the final lipid concentration is at least or about 5 mM to about 20 mM. In yet another embodiment, the final lipid concentration is at least or about 10 mM to about 15 mM.

In yet a further embodiment, the mixing solution lipid:nucleic acid weight ratio is at least or about 1:0, 2:1, 3:1, 4:1, 5:1, 6:1, 7:1, 8:1, 9:1, 10:1, 11:1, 12:1, 15:1, 17:1, 18:1, 20:1, 25:1, 30:1, 35:1, 40:1 or 50:1.

In yet a further embodiment, the LNP is made using a nucleic acid concentration less than 0.2 mg/ml. In another embodiment, the LNP is made using a final lipid concentration less than 10 mM. In yet another embodiment, the LNP is made using a nucleic acid concentration less than 0.2 mg/ml and a final lipid concentration less than 5 mM. In still another embodiment, the potency is about 1.5 times more than the LNP formed using higher lipid concentrations.

In various embodiments, the method does not include a dialysis step.

In another embodiment, the method comprises diluting an LNP solution using a buffer solution to obtain an ethanol concentration of below about 350 mM. In another embodiment, the method comprises diluting an LNP solution using a buffer solution to obtain an ethanol concentration of below about 300 mM. In another embodiment, the method comprises diluting an LNP solution using a buffer solution to obtain an ethanol concentration of below about 250 mM. In another embodiment, the method comprises diluting an LNP solution using a buffer solution to obtain an ethanol concentration of below about 200 mM. In another embodiment, the method comprises diluting an LNP solution using a buffer solution to obtain an ethanol concentration of below about 150 mM. In another embodiment, the method comprises diluting an LNP solution using a buffer solution to obtain an ethanol concentration of below about 140 mM. In another embodiment, the method comprises diluting an LNP solution using a buffer solution to obtain an ethanol concentration of below about 130 mM. In another embodiment, the method comprises diluting an LNP solution using a buffer solution to obtain an ethanol concentration of below about 120 mM. In another embodiment, the method comprises diluting an LNP solution using a buffer solution to obtain an ethanol concentration of below about 110 mM. In another embodiment, the method comprises diluting an LNP solution using a buffer solution to obtain an ethanol concentration of below about 100 mM. In another embodiment, the method comprises diluting an LNP solution using a buffer solution to obtain an ethanol concentration below about 90 mM. In another embodiment, the method comprises diluting an LNP solution using a buffer solution to obtain an ethanol concentration of below about 80 mM. In another embodiment, the method comprises diluting an LNP solution using a buffer solution to obtain an ethanol concentration below about 70 mM. In another embodiment, the method comprises diluting an LNP solution using a buffer solution to obtain an ethanol concentration of below about 60 mM. In another embodiment, the method comprises diluting an LNP solution using a buffer solution to obtain an ethanol concentration of below about 50 mM. In another embodiment, the method comprises diluting an LNP solution using a buffer solution to obtain an ethanol concentration of below about 40 mM. In another embodiment, the method comprises diluting an LNP solution using a buffer solution to obtain an ethanol concentration of below about 30 mM. In another embodiment, the method comprises diluting an LNP solution using a buffer solution to obtain an ethanol concentration of below about 30 mM. In another embodiment, the method comprises diluting an LNP solution using a buffer solution to obtain an ethanol concentration of below about 20 mM. In another embodiment, the method comprises diluting an LNP solution using a buffer solution to obtain an ethanol concentration of below about 10 mM. In another embodiment, the method comprises diluting an LNP solution using a buffer solution to obtain an ethanol concentration of below about 5 mM. In another embodiment, the method comprises diluting an LNP solution using a buffer solution to obtain an ethanol concentration of below about 1 mM.

In another embodiment, the potency of an LNP formed after dilution with a buffer to obtain an ethanol concentration below about 350 mM is about 1.2 to about 2 times more than an LNP formed with a higher concentration and using dialysis to remove organic solvent (e.g., ethanol). In another embodiment, the potency of an LNP formed after dilution with a buffer to obtain an ethanol concentration below about 300 mM is about 1.2 to about 2 times more than an LNP formed with a higher concentration and using dialysis to remove organic solvent (e.g., ethanol). In another embodiment, the potency of an LNP formed after dilution with a buffer to obtain an ethanol concentration below about 250 mM is about 1.2 to about 2 times more than an LNP formed with a higher concentration and using dialysis to remove organic solvent (e.g., ethanol). In another embodiment, the potency of an LNP formed after dilution with a buffer to obtain an ethanol concentration below about 200 mM is about 1.2 to about 2 times more than an LNP formed with a higher concentration and using dialysis to remove organic solvent (e.g., ethanol). In another embodiment, the potency of an LNP formed after dilution with a buffer to obtain an ethanol concentration below about 150 mM is about 1.2 to about 2 times more than an LNP formed with a higher concentration and using dialysis to remove organic solvent (e.g., ethanol). In another embodiment, the potency of an LNP formed after dilution with a buffer to obtain an ethanol concentration below about 100 mM is about 1.2 to about 2 times more than an LNP formed with a higher concentration and using dialysis to remove organic solvent (e.g., ethanol). In another embodiment, the potency of an LNP formed after dilution with a buffer to obtain an ethanol concentration below about 75 mM is about 1.2 to about 2 times more than an LNP formed with a higher concentration and using dialysis to remove organic solvent (e.g., ethanol). In another embodiment the potency of an LNP formed after dilution with a buffer to obtain an ethanol concentration below about 50 mM is about 1.2 to about 2 times more than an LNP formed with a higher concentration and using dialysis to remove organic solvent (e.g., ethanol). In another embodiment, the potency of an LNP formed after dilution with a buffer to obtain an ethanol concentration below about 25 mM is about 1.2 to about 2 times more than an LNP formed with a higher concentration and using dialysis to remove organic solvent (e.g., ethanol). In another embodiment, the potency of an LNP formed after dilution with a buffer to obtain an ethanol concentration below about 10 mM is about 1.2 to about 2 times more than an LNP formed with a higher concentration and using dialysis to remove organic solvent (e.g., ethanol). In another embodiment, the potency of an LNP formed after dilution with a buffer to obtain an ethanol concentration below about 50 mM is about 1.2 to about 2 times more than an LNP formed with a higher concentration and using dialysis to remove organic solvent (e.g., ethanol). In another embodiment, the potency of an LNP formed after dilution with a buffer to obtain an ethanol concentration below about 25 mM is about 1.2 to about 2 times more 993 than an LNP formed with a higher concentration and using dialysis to remove organic solvent (e.g., ethanol).

In one embodiment of the invention, the LNPs have an average diameter in the range of about 40 to about 150 nanometers. In another embodiment, the LNPs have an average diameter in the range of about 50 to about 100 nanometers. In a further embodiment, the LNPs have a nucleic acid encapsulation efficiency of about 60 to about 95%. In still another embodiment, the LNPs have a nucleic acid encapsulation efficiency of about 70% to about 90%. In still another embodiment, the LNPs have a nucleic acid encapsulation efficiency of about 75% to about 85%. In still a further embodiment, the LNPs have a nucleic acid encapsulation efficiency of about 78% to about 83%.

Another aspect of the invention relates to a pharmaceutical composition comprising the LNPs made according to the methods described herein. In one embodiment, the pharmaceutical composition is a vaccine. In another embodiment, the vaccine is prophylactic. In further embodiment, the vaccines is a therapeutic vaccine. In another embodiment, the vaccine is to treat or prevent an infectious disease. In a further embodiment, the vaccine is a veterinary vaccine. In a further embodiment, the composition comprises a bioactive agent selected from the group consisting of a peptide, antibody, antibody fragment, and small molecule therapeutics.

### DESCRIPTION OF THE FIGURES DRAWINGS

A more complete understanding of the presently disclosed subject matter can be obtained by reference to the accompanying Figures when considered in conjunction with the subsequent detailed description. The embodiments illustrated in the Figures are intended to be exemplary only and should not be construed as limiting the presently disclosed subject matter to the illustrated embodiments. The figures described below are associated with experimental results from the various Examples described below.

FIGS. 1A, 1B, and 1C illustrate flow cytometry data 24 hours after transfection of cells with a 0.25 μg dose of saRNA in BHK cells. Exemplary embodiments are provided illustrating the percentage of cells that expressed GFP protein in untransfected cells (−) Control (FIG. 1A), saRNA transfected with Mirus transfection kit (+) Control (FIG. 1B), or saRNA-LNP vaccine made by direct dilution (FIG. 1C). FIGS. 2A, 2B, and 2C illustrate flow cytometry data 24 hours after transfection of cells with a 0.25 μg dose of saRNA in HEK cells. Exemplary embodiments are provided illustrating the percentage of cells that expressed GFP protein in untransfected cells (−) Control (FIG. 2A), saRNA transfected with Mirus transfection kit (+) Control (FIG. 2B), or saRNA-LNP vaccine made by direct dilution (FIG. 2C).

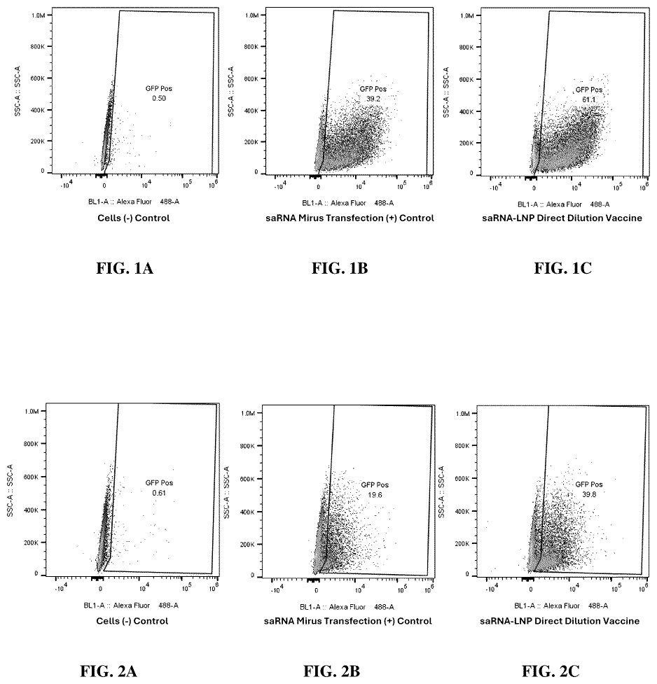

### DETAILED DESCRIPTION OF THE INVENTION

Disclosed herein are methods of increasing the potency of nucleic acid-lipid nanoparticles through a novel and superior LNP manufacturing process.

The methods disclosed herein overcome technical difficulties and high costs associated with previous LNP manufacturing techniques that use dialysis to remove ethanol. The method disclosed herein, therefore, greatly reduces the cost of industrial production of saRNA-LNPs.

One embodiment of the invention disclosed herein is a method that increases the potency of saRNA-LNPs by eliminating the loss of saRNA integrity that occurs during dialysis.

The methods disclosed herein are applicable to any ionizable lipid and an saRNA payload. While not desiring to be bound by any particular mechanism of action, increased LNP potency is believed to be mediated through a more intact saRNA-LNP, increased endosomal release and subsequent dissociation of the saRNA from the ionizable lipid. Preferably, LNPs delivering nucleic acids, e.g., saRNA encoded immunogens, formed by the methods disclosed herein will be more potent e.g., providing greater protection against viral challenge, compared to those formed at current methods that utilize dialysis.

All technical and scientific terms used herein, unless otherwise defined below, are intended to have the same meaning as commonly understood by one of ordinary skill in the art. Mention of techniques employed herein is intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate an explanation of the presently disclosed subject matter. Thus, unless defined otherwise, all technical and scientific terms and any acronyms used herein have the same meanings as commonly understood by one of ordinary skill in the art in the field of the presently disclosed subject matter. Although any compositions, methods, kits, and means for communicating information similar or equivalent to those described herein can be used to practice the presently disclosed subject matter, particular compositions, methods, kits, and means for communicating information are described herein. It is understood that the particular compositions, methods, kits, and means for communicating information described herein are exemplary only and the presently disclosed subject matter is not intended to be limited to just those embodiments.

Following long-standing patent law convention, the terms “a”, “an”, and “the” refer to “one or more” when used in this application, including the claims. For example, in some embodiments the phrase “an LNP,” a “nucleic acid,” refers to one or more LNPs or nucleotides, respectively.

It should be understood that for all numerical bounds describing some parameter in this application, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

The term “about”, as used herein to refer to a measurable value such as an amount of weight, time, dose (e.g., therapeutic dose), etc., is meant to encompass in some embodiments variations of +/−20%, in some embodiments +/−10%, in some embodiments +/−5%, in some embodiments +/−1%, in some embodiments +/−0.1%, in some embodiments +/−0.5%, and in some embodiments +/−0.01% from the specified amount, as such variations are appropriate to perform the disclosed methods.

As used herein, the term “and/or” when used in the context of a list of entities, refers to the entities being present singly or in any and every possible combination and subcombination. Thus, for example, the phrase “A, B, C, and/or D” includes A, B, C, and D individually, but also includes any and all combinations and subcombinations of A, B, C, and D. It is further understood that for each instance wherein multiple possible options are listed for a given element (i.e., for all “Markush Groups” and similar listings of optional components for any element), in some embodiments the optional components can be present singly or in any combination or subcombination of the optional components. It is implicit in these forms of lists that each and every combination and subcombination is envisioned and that each such combination or subcombination has not been listed simply merely for convenience. Additionally, it is further understood that all recitations of “or” are to be interpreted as “and/or” unless the context clearly requires that listed components be considered only in the alternative (e.g., if the components would be mutually exclusive in a given context and/or could not be employed in combination with each other).

The term “lipid” refers to a group of organic compounds that are esters of fatty acids and are characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

The term “lipid nanoparticle” or “LNP” used herein refers to a particle that comprises a plurality of, i.e. more than one, lipid molecules physically associated with each other by intermolecular forces. In one embodiment, the LNP carries a nucleic acid payload. The LNPs can have one or more different types of lipids. The lipid nanoparticles may be, e.g., microspheres (including unilamellar and multilamellar vesicles, e.g., “liposomes”-lamellar phase lipid bilayers that, in some embodiments are substantially spherical, and, in more particular embodiments can comprise an aqueous core, e.g., comprising a substantial portion of RNA molecules), a dispersed phase in an emulsion, micelles or an internal phase in a suspension.

In some embodiments, the lipid nanoparticles have a size of about 1 to about 2,500 nm, about 10 to about 1,500 nm, about 20 to about 1,000 nm, in one embodiment about 50 to about 600 nm, in a sub-embodiment about 50 to about 400 nm, in a sub-embodiment about 50 to about 250 nm, and in a sub-embodiment about 50 to about 150 nm. Unless indicated otherwise, all sizes referred to herein are the fully formed nanoparticle's average sizes (diameters), as measured by dynamic light scattering on a Malvern Zetasizer. The nanoparticle sample is diluted in phosphate-buffered saline (PBS) so that the count rate is approximately 200-400 kcts. The data are presented as the number-weighted average obtained by transformation of the intensity-weighted average. The number-weighted average is preferred since it most closely corresponds to the physical diameter of the particle as measured by electron microscopy.

“LNP lipid” as used herein refers to the individual lipid molecules that form an LNP. In certain embodiments, the LNP lipids are ionizable cationic lipids.

As used herein, the term “cationic lipid” refers to a lipid that is cationic or becomes cationic (protonated) as the pH is lowered below the pKa of the ionizable group of the lipid when present in the LNP (i.e. the pKa of the ionizable lipid in the lipid environment of the LNP which is different from the pKa of the ionizable lipid in aqueous media), but is progressively more neutral at higher pH values. At pH values below the pKa, the lipid is then able to associate with negatively charged nucleic acids (e.g., oligonucleotides). As used herein, the term “cationic lipid” includes zwitterionic lipids that assume a positive charge on pH decrease. Notably most helper lipids such as DSPC are zwitterionic but not cationic since they have phosphate groups which balance any cationic charge.

The term “cationic lipid” also refers to any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy) propyl)-N,N,N-trimethylammonium chloride (DOTMA); N,N-distearyl-N,N-dimethylammonium bromide (DDAB); N-(2,3-dioleoyloxy) propyl)-N,N,N-trimethylammonium chloride (DOTAP); 3-(N—(N′,N′-dimethylaminoethane)-carbamoyl) cholesterol (DC-Chol) and N-(1,2-dimyristyloxyprop-3-yl)-N,N-dimethyl-N-hydroxyethyl ammonium bromide (DMRIE). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy) propyl)-N-(2-(sperminecarboxamido)ethyl)-N,N-dimethyl-ammonium trifluoroacetate (DOSPA) and (DOPE), from GIBCO/BRL); and TRANSFECTAM®. (Commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

The inventive LNP compositions disclosed herein can include one or more biologically active agents including, but not limited to, antibodies (e.g., monoclonal, chimeric, humanized, nanobodies, and fragments thereof etc.), cholesterol, hormones, peptides, proteins, chemotherapeutics and other types of antineoplastic agents, low molecular weight drugs, vitamins, co-factors, nucleosides, nucleotides, oligonucleotides, and enzymatic nucleic acids.

The term “nucleic acids” refers to ribonucleotides, deoxynucleotides, modified ribonucleotides, modified deoxyribonucleotides, modified phosphate-sugar-backbone oligonucleotides, other nucleotides, nucleotide analogs, and combinations thereof, and can be single stranded, double stranded, or contain portions of both double stranded and single stranded sequence, as appropriate. In some embodiments, “nucleic acid” includes antisense nucleic acids, triplex forming oligonucleotides, antisense DNA or RNA compositions, chimeric DNA: RNA compositions, allozymes, aptamers, ribozyme, decoys and analogs thereof, plasmids and other types of expression vectors, and small nucleic acid molecules, RNAi agents, self-amplifying RNA (saRNA), short interfering nucleic acid (siNA), messenger ribonucleic acid” (messenger RNA, mRNA), short interfering RNA (siRNA), double-stranded RNA (dsRNA), micro-RNA (miRNA), and short hairpin RNA (shRNA) molecules, peptide nucleic acid (PNA), a locked nucleic acid ribonucleotide (LNA), morpholino nucleotide, threose nucleic acid (TNA), glycol nucleic acid (GNA), sisiRNA (small internally segmented interfering RNA), aiRNA (asymmetrical interfering RNA), and siRNA with 1, 2 or more mismatches between the sense and anti-sense strand to relevant cells and/or tissues, such as in a cell culture, subject or organism. Such compounds may be purified or partially purified, and may be naturally occurring or synthetic, and may be chemically modified. In one embodiment the biologically active agent is an RNAi agent, short interfering nucleic acid (siNA), short interfering RNA (siRNA), double-stranded RNA (dsRNA), micro-RNA (miRNA), or a short hairpin RNA (shRNA) molecule. In one embodiment the biologically active agent is a RNAi agent useful for mediating RNA interference (RNAi). In a preferred embodiment, the biologically active agent is saRNA.

As used herein, the term “nucleic acid” is also meant to include any oligonucleotide or polynucleotide. Fragments containing up to 50 nucleotides are generally termed oligonucleotides, and longer fragments are called polynucleotides. In particular embodiments, oligonucleotides of the present invention are 20-50 nucleotides in length. In the context of this invention, the terms “polynucleotide” and “oligonucleotide” refer to a polymer or oligomer of nucleotide or nucleoside monomers consisting of naturally occurring bases, sugars and intersugar (backbone) linkages. The terms “polynucleotide” and “oligonucleotide” also includes polymers or oligomers comprising non-naturally occurring monomers, or portions thereof, which function similarly. Such modified or substituted oligonucleotides are often preferred over native forms because of properties such as, for example, enhanced cellular uptake and increased stability in the presence of nucleases. Oligonucleotides are classified as deoxyribooligonucleotides or ribooligonucleotides. A deoxyribooligonucleotide consists of a 5-carbon sugar called deoxyribose joined covalently to phosphate at the 5′ and 3′ carbons of this sugar to form an alternating, unbranched polymer. A ribooligonucleotide consists of a similar repeating structure where the 5-carbon sugar is ribose. The nucleic acid that is present in a lipid particle according to this invention includes any form of nucleic acid that is known. The nucleic acids used herein can be single-stranded DNA or RNA, or double-stranded DNA or RNA, or DNA-RNA hybrids. Examples of double-stranded DNA include structural genes, genes including control and termination regions, and self-replicating systems such as viral or plasmid DNA. Examples of double-stranded RNA include siRNA and other RNA interference reagents. Single-stranded nucleic acids include antisense oligonucleotides, ribozymes, microRNA, and triplex-forming oligonucleotides.

In another embodiment the biologically active agent is saRNA.

“Potency” as referred to herein refers to the ability of an LNP to deliver a nucleic acid payload to a cell or tissue, where the LNP is internalized into the cell (or cells in a tissue) and released from the endosome to the cytoplasm whereupon the nucleic acid payload is released from the lipid and becomes bioavailable. Potency may be measured in any number of ways known to those of skill in the art. For example, it may be measured in terms of cell uptake, percentage of encapsulation, nucleic acid payload transcription, nucleic acid payload translation, or production of a polypeptide encoded by the nucleic acid payload. Where an LNP's nucleic acid payload is meant to function in a gene expression inhibitory manner such as in for example in RNAi, LNP potency may be measured in terms of target gene ‘knock down’ through reduction of the target gene's transcription rate, the length of the gene target's mRNA transcripts half-life, or translation of the target gene's mRNA transcript. Additional assays for measuring potency depend on measuring LNP immunogenicity and LNP systemic distribution. All such assays and their permutations are well known in the art.

On one embodiment an experiment measuring LNP potency is conducted in parallel to a reference LNP. Such experiments may use a standardized nucleic acid payload such as, for example, a reporter gene. A reporter gene (often simply reporter) is a gene that researchers attach to a regulatory sequence of another gene of interest in bacteria, cell culture, animals, or plants. Such genes are called reporters because the characteristics they confer on organisms expressing them are easily identified and measured, or because they are selectable markers. Reporter genes may be used as an indication of whether a certain gene has been taken up by or expressed in the cell or organism population. Typical reporters genes are lacZ, cat, gfp, rfp, luc, which encode β-galactosidase, Chloramphenicol acetyltransferase, Green fluorescent protein, Red fluorescent protein, Luciferase enzyme, respectively, which can be used in respective histochemical, acetylation, fluorescent, spectrophotometric, and bioluminescence assays. All such assays and their permutations are well known in the art.

In one embodiment, LNP Potency is measured by Luciferase reporter activity in vitro or in vivo at a known dose or several doses. Relative LNP potency is determined by Luciferase activity measure in vitro or in vivo. See also for example, U.S. Pat. No. 10,221,127.

LNP Potency may also be measured in terms of a desired biological reaction to the nucleic acid payload including for example, a therapeutic or prophylactic effect or an impact on a mechanism of action leading thereto. In some embodiments, LNP potency is measured by the ability of an LNP carrying an mRNA encoding an immunogen, e.g., a polypeptide that induces the immune system to make a secreted cognate IgG antibody after administration.

“Increase in LNP Potency” refers to the extent to which the inventive LNP formed by diluting the concentration of ethanol using a buffer has greater potency than a LNP formed using a high concentration of organic solvent requiring removal by dialysis. In certain embodiments, the inventive LNPs disclosed herein has an increase in LNP Potency that is about or at least a factor of 1.25, 1.50, 1.75, or 2 times greater than a LNP in the same assay delivering the same nucleic acid cargo.

“Organic solvents” as used herein refers to a type of volatile organic compound (VOC). VOCs are organic chemicals which vaporize at room temperature and are typical in the art for dissolving certain materials and substances in the manufacture of pharmaceutical products. Organic solvents in manufacturing are typically used include alcohols, e.g., methanol, ethanol; esters; and ethers, e.g., acetone, amines, nitrated and halogenated hydrocarbons.

In one embodiment, the amount of nucleic acid is represented by the LNP mole ratio of the amine on the ionizable lipid to the phosphate groups on the nucleic acid backbone and is typically about 3 to about 12. In one embodiment, the pKa of the LNP is in the about 6 to about 7 range corresponding to the pH in the early endosome. A link between the pKa of ionizable lipid in the LNP and gene silencing efficiency has shown that an LNP pKa in the range of about 6 to about 7 produced more silencing for the ionizable lipid DLinDMA and was associated with promoting lipid structures that could disrupt the membrane of the endosome. The pKa of the LNP may be measured using the pH-dependence of fluorescence enhancement of the anionic dye TNS.

As used herein “cholesterol” refers to a biologically active organic compound with four rings arranged in a specific molecular configuration. The steroid core structure is typically composed of seventeen carbon atoms, bonded in four “fused” rings: three six-member cyclohexane rings (rings A, B and C) and one five-member cyclopentane ring (the D ring). Steroids vary by the functional groups attached to this four-ring core and by the oxidation state of the rings. Sterols are forms of steroids with a hydroxy group at position three and a skeleton derived from cholestane. Steroids can also be more radically modified, such as by changes to the ring structure, for example, cutting one of the rings. Cutting Ring B produces secosteroids one of which is vitamin D3. Examples include the lipid cholesterol, the sex hormones estradiol and testosterone, [4]: 10-19 and the anti-inflammatory drug dexamethasone. Many steroids are found in plants, animals and fungi. Steroids are preferably manufactured in cells from the sterols lanosterol or cycloartenol (plants). Lanosterol and cycloartenol are derived from the cyclization of the triterpene squalene. In some embodiments, the LNPs compositions disclosed herein contain a cholesterol derivative which is for example, dihydrocholesterol, ent-cholesterol, epi-cholesterol, desmosterol, cholestanol, cholestanone, cholestenone, cholesteryl-2′-hydroxyethyl ether, cholesteryl-4′-hydroxybutyl ether, 3.beta.-[N—(N′N′-dimethylaminoethyl) carbamoyl cholesterol (DC-Chol), 24 (S)-hydroxycholesterol, 25-hydroxycholesterol, 25 (R)-27-hydroxycholesterol, 22-oxacholesterol, 23-oxacholesterol, 24-oxacholesterol, cycloartenol, 22-ketosterol, 20-hydroxysterol, 7-hydroxycholesterol, 19-hydroxycholesterol, 22-hydroxycholesterol, 25-hydroxycholesterol, 7-dehydrocholesterol, 5.alpha.-cholest-7-en-3.beta.-ol, 3,6,9-trioxaoctan-1-ol-cholesteryl-3e-ol, dehydroergosterol, dehydroepiandrosterone, lanosterol, dihydrolanosterol, lanostenol, lumisterol, sitocalciferol, calcipotriol, coprostanol, cholecalciferol, lupeol, ergocalciferol, 22-dihydroegocalciferol, ergosterol, brassicasterol, tomatidine, tomatine, ursolic acid, cholic acid, chenodeoxycholic acid, zymosterol, diosgenin, fucosterol, fecosterol, or fecosterol, or a salt or ester thereof. In some embodiments, the cholesterol or cholesterol derivative is cholesterol, cholesterol succinic acid, cholesterol sulfate, cholesterol hemisuccinate, cholesterol phthalate, cholesterol phosphate, cholesterol valerate, cholesterol acetate, cholesteryl oleate, cholesteryl linoleate, cholesteryl myristate, cholesteryl palmitate, cholesteryl arachidate, cholesteryl phosphorylcholine, and sodium cholate. Other exemplary steroids are disclosed in U.S. Publication No. 20200129445.

As used herein, “lipid encapsulated” refers to a lipid nanoparticle that provides an active agent or therapeutic agent, such as a nucleic acid (e.g., mRNA), with full encapsulation, partial encapsulation, or both. In an embodiment, the nucleic acid (e.g., mRNA) is fully encapsulated in the LNP.

### Methods of LNP Manufacture

### Current State-of-the-Art Methods Using Dialysis

The prior art has heretofore utilized a method for LNP manufacture that included dialysis to remove ethanol resulting in high cost of producing RNA-based vaccines, poor recovery and loss of RNA integrity during manufacture, and lack of stability during storage.

Indeed, the Standard Manufacturing Method that Includes Dialysis may be summarized as follows:
Nucleic Acid Solution: Prepare saRNA at a concentration of about less than 0.20 mg/ml in a buffer at one concentration and one pH, i.e. 50 mM citrate pH 5 or 25 mM sodium acetate pH 4, or 10-50 mM citrate pH 4.
Lipid Solution: Prepare lipid mix in ethanol at a concentration corresponding to the desired ionizable cationic lipid/mRNA weight ratio (3:1 to 30:1)
Assemble LNPs: Combining portions of the nucleic acid solution and portions of the Lipid Solution result in a single mixing solution in a buffer.

Dialyze nucleic acid-LNPs in the mixing solution to physiological pH.

Measure nucleic acid-LNP size using light scattering and nucleic acid by absorbance or fluorescence.

Measure nucleic acid encapsulation in the LNPs using the Ribogreen assay.

Select nucleic acid—LNP preparations that have high encapsulation >70% and diameters from 50-100 nm.

“Mixing” preferably using turbulent mixing (“T-mix”), vortex mixing (“V-mix”), microfluidic mixing, or both. See for example mixing described in U.S. Pub. No. 20200306191, incorporated by reference.

In one embodiment, the Standard LNP Manufacturing Method is that described in Hassett et al., Mol. Ther.-Nucleic Acids 2019, 15, 1-11. In another embodiment, the Reference Manufacturing Method” is as described in U.S. App. Ser. No. 20190032087. In one embodiment, the reference method includes: (a) introducing a first stream comprising a therapeutic agent (e.g., nucleic acid) in a first solvent into a microchannel; wherein the microchannel has a first region adapted for flowing one or more streams introduced into the microchannel and a second region for mixing the contents of the one or more streams; (b) introducing a second stream comprising LNP-forming materials (e.g., reference LNP lipids) in a second solvent in the microchannel to provide first and second streams flowing under laminar flow conditions, wherein the lipid particle-forming materials comprise an ionizable lipid, and wherein the first and second solvents are the same or are not the same; (c) flowing the one or more first streams and the one or more second streams from the first region of the microchannel into the second region of the microchannel; and (d) mixing of the contents of the one or more first streams and the one or more second streams in the second region of the microchannel to provide a third stream comprising lipid particles with encapsulated therapeutic agents. The contents of the first and second streams can be mixed by chaotic advection in microfluidic channels or by nanoprecipitation in a T-mixer.

Mixing the contents of the one or more first streams and the one or more second streams comprise varying the concentration or relative mixing rates of the one or more first streams and the one or more second streams. To further stabilize the third stream containing the lipid particles with encapsulated therapeutic agents, the method can, but need not further include, comprising diluting the third stream with an aqueous buffer. Diluting the third stream includes flowing the third stream and an aqueous buffer into a second mixing structure. The aqueous buffer comprising lipid particles with encapsulated therapeutic agents is dialyzed to reduce the amount of the organic solvent.

### Methods Making LNPs of the Invention

The claimed invention encompasses a method that does not remove the organic solvent using dialysis but rather dilutes the concentration to provide a safe concentration of the organic solvent (e.g., ethanol). In various embodiments, the inventors surprisingly found that lowering ethanol to a non-toxic concentration by dilution with a cryoprotectant buffer produced saRNA-LNPs with much greater yield and potency and, therefore, at much less cost than saRNA-LNPs prepared with dialysis.

The inventors further submit that it was heretofore unknown that a dilution to a non-toxic level of ethanol could be used to prepare saRNA-LNPs that are suitable for in vivo administration.

Disclosed herein is the unexpected finding that LNP potency is strongly affected by reducing the ethanol concentration by dilution rather than dialysis.

In general embodiments, the invention encompasses an enhanced method to manufacture a lipid nanoparticle, wherein the lipid nanoparticle delivery efficiency depends on dilution of the saRNA-LNP organic solvent concentration and avoids dialysis.

In various embodiments, the LNPs with high delivery efficiency are critically important in the success of saRNA vaccines. In certain embodiments, the LNPs contain 4 lipids, an ionizable lipid; the helper lipid, DSPC; cholesterol; and a PEG lipid that self-assemble with the saRNA sequence into a nanoparticle with diameter of ˜60 to 150 nm. In certain embodiments, the protonated form of the ionizable lipid electrostatically binds the anionic phosphate backbone of the saRNA to encapsulate it in the LNP while DSPC forms a peripheral bilayer that contains the PEG-lipid tail with the hydrophilic PEG domain facing the aqueous medium. In certain embodiments, the role of the ionizable lipid is to facilitate endosomal release by protonating as the endosomal pH drops below 7 and then interacting with the endosomal membrane to open it and release the saRNA.

In certain embodiments, the delivery efficiency depends on the saRNA-LNP manufacturing process. In certain embodiments, the saRNA-LNPs are manufactured using a microfluidic or a larger scale T-mixer through a self-assembly process where the 4 lipids in ethanol are mixed rapidly with the mRNA that is in a low pH buffer. In certain embodiments, the formation of the saRNA-LNP occurs through electrostatic binding of the protonated cationic ionizable lipid with the anionic saRNA phosphate backbone followed by the lipids segregating from the aqueous phase to form the nanoparticle that is stabilized by the hydrophilic PEG interface.

In general embodiments, improved methods for making LNPs disclosed herein may be summarized as follows:
Nucleic Acid Solution: Provide nucleic acids at a Nucleic Acid concentration in a buffer at a certain buffer concentration and at a certain Nucleic Acid Solution pH.
Lipid Solution: Provide lipids in an organic solvent at a Lipid Solution lipid concentration.
Assemble LNPs: Combine portions of the Nucleic Acid Solution and Lipid Solution into a Mixing Solution having a mixing buffer concentration and pH.
Dilution: Dilute the Nucleic Acid-LNP Solution using a cryoprotectant buffer to obtain an ethanol concentration of less than 350 mM.

The skilled artisan will understand the certain steps in the inventive methods need not be performed in a certain order while other steps must be performed before others. Moreover, various parties might perform various steps of the overall method.

In some embodiments, the portions of the Nucleic Acid Solution and the Lipid Solution in the Mixing Solution are in volume ratio are about or at least 0.1:1, 0.2:1, 0.3:1, 0.4:1, 0.5:1, 0.6:1, 0.7:1, 0.8:1, 0.9:1, 1:1, 2:1, 3:2, 3:1, 4:1, 4:3, 5:1, 5:3, 5:4, 6:1, 6:5, 7:1, 8:1, 9:10, 10:1, 11:1, 12:1, 13:1, 14:1, 15:1, 16:1, 17:1, 18:1, 19:1, and 20:1.

LNPs made according to the method above may then be further processed for use according to methods well known in the art. In some embodiments, such further processing involves one or more of the following:
Measuring LNP size, e.g., by light scattering.
Measuring RNA encapsulation and concentration, e.g., by Ribogreen assay.
Selecting LNPs that have high encapsulation, e.g., at least or about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent encapsulation efficiency; or an average diameter at least or about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 175, 200, 250, 300, 350, 400, 450, 500 nM; or having a minimum diameter of about 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 nM and a maximum diameter of about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 175, 200, 250, 300, 350, 400, 450, 500 nM, respectively.

In some embodiments, the Nucleic Acid Solution contains nucleic acids at a nucleic acid concentration of about or at least 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 milligrams/ml.

In some embodiments, the nucleic acids are present in about or at least 1, 2, 3, 4, 5, 6, 7, 8, 9, 10, 11, 12, 13, 14, 15, 16, 17, 18, 19, 20, or more molecular species each of which encode at about or at least 1, 2, 3, 4, 5, 6, 7, 8, 9, 10, 11, 12, 13, 14, 15, 16, 17, 18, 19, 20, or more open reading frames.

In some embodiments, the nucleic acid molecule can be about or at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 50, 100, 200, 300, 400, 500, 750, 800, 1000, 2000, 3000, 4000, 5000, 6000, 7000, 8000, 9000, 10000, 11000, 15000, 20000, 30000, 40000, 50000, 75000, 100000 nucleotides in length.

In some embodiments, the Nucleic Acid Solution contains a buffer at a concentration of about or at least 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 175, 200, 250, 300, 350, 400, 450, 500 mM.

In some embodiments the buffer is salt buffer. In some embodiments, the buffering agent is citric acid, acetic acid, phosphate, and borate. In some embodiments, the buffer is potassium, magnesium, or sodium acetate. In other embodiments, the buffer may be sucrose, citrate, MES, Histidine, ADA, ACES, PIPES, MOPSO, BES, HEPES, DIPSO, TEA, AMPD, Gly-Gly, TAPS, HEPBS, AMPD, TABS, AMP, CAPSO, CAPS, CABS, CHES, PBS, SSC, TAE, TBE, or TE and the like. See for example, “Acetate Buffer (pH 3.6 to 5.6) Preparation.” AAT Bioquest, Inc, 29 Sep. 2020.

In some embodiments, the Nucleic Acid Solution is at a pH of about or at least 3, 3.5, 4, 4.5, 5, 5.5, 6, 6.5, 7, or 7.5.

In some embodiments, the Lipid Solution contains the organic solvent is alcohols, e.g., methanol or ethanol, esters, ethers, ketones, e.g., acetone, amines, nitrated and/or halogenated hydrocarbons; or a combination thereof. In one embodiment the organic solvent is ethanol. Preferred solvents are volatile, non-toxic, and/or acceptable for administration to humans in low amounts.

In some embodiments, the Lipid Solution contains a one or more lipids at a total concentration of about or less than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mM.

In some embodiments, Mixing Solution has a mixing concentration of nucleic acid of about or at least 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2 milligrams/ml.

In some embodiments, Mixing Solution has a mixing total concentration of lipids of about or less than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mM

In some embodiments, Mixing Solution has a mixing buffer concentration of about or at least 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 175, 200, 250, 300, 350, 400, 450, 500 mM.

In some embodiments, the Mixing Solution is at a pH of about or at least 3, 3.5, 4, 4.5, 5, 5.5, 6, 6.5, 7, 7.5, or 8.

In some embodiments the desired “) lipid/nucleic acid weight ratio is about: 1:1 to 1000:1, preferably 5:1-100:1, more preferably 10:1-30:1.

In some embodiments, the final Nucleic Acid-LNP Solution is diluted using a buffer solution, for example a cryoprotectant buffer to a final ethanol concentration of less than about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 125, 150, 200, 250, 300 or about 350 mM.

In one embodiment, a Methods of Manufacture is as set for the below:
Nucleic Acid Solution: Prepare saRNA at several concentrations in the range 0.05 to 3 mg/ml in a single buffer, e.g. 25 mM sodium acetate buffer at pH 4.
Lipid Solution: Prepare the lipid mix in ethanol or another suitable solvent at several concentrations for each of the mRNA solutions described immediately above in relation to the Nucleic Acid Solution preparation.
Assemble LNPs at the above multiple mixing concentrations with saRNA in a single buffer type, concentration and pH.
Adjust the saRNA-LNP solution to physiological pH.
Dilute the saRNA-LNP solution with cryoprotectant buffer to the desired saRNA concentration.
Measure the saRNA concentration and the LNP encapsulation percentage using the Ribogreen assay. Accept LNPs that have encapsulation >40%.
Measure saRNA-LNP size using light scattering.
Calculate the ethanol concentration produced by the dilution of the starting saRNA-LNP solution.
Measure Luciferase activity in vitro or in vivo at a known dose or several doses.
Determine saRNA-LNP potency by Luciferase expression in vitro.

In another embodiment LNP potency may be optimized at any given mixing concentration by changing the pH of mixing and protonation level of the ionizable lipid during mixing by adjusting the RNA buffer type, concentration and pH:
Nucleic Acid Solution: Prepare saRNA at one concentration (i.e. in the above range 0.05 to 3 mg/ml) in a range of buffer types (sodium acetate, sodium citrate etc.), buffer concentrations (1-100 mM) and pH (3-7). For any particular mixing concentration, the buffers chosen should produce encapsulation efficiencies spanning the range of 40-90% which could correspond approximately to the protonation level (40-90%) of the ionizable lipid which in turn is determined by the pH of the mixing buffer and thus the buffer type, concentration and pH.
Lipid Solution: Prepare the lipid mix in ethanol or another suitable solvent at a concentration less than 50 mM.
Assemble LNPs in the multiple buffers above with a single mixing concentration.
Final Nucleic Acid-LNP Solution-Bringing Nucleic Acid-LNPs to physiological pH and dilute using a buffering solution to various concentrations.
Measure LNP size using light scattering
Measure RNA encapsulation in the LNPs using the Ribogreen assay
Accept LNPs that have encapsulation >80%
Determine saRNA-LNP potency by measuring Luciferase expression in vitro.

TABLE 1
Ethanol concentration of the Dialysis
and Direct-Dilution Formulations

| Method | saRNA (μg/mL) | Ethanol Conc % (v/v) |
|---|---|---|
| Dialysis | 57 | 0.06 |
| Direct Dilution | 16 | 4.38 |
| Direct Dilution | 8 | 1.73 |
| Direct Dilution | 4 | 0.89 |
| Direct Dilution | 2 | 0.45 |
| Direct Dilution | 1 | 0.21 |
| Direct Dilution | 0.5 | 0.08 |

TABLE 2
LNPs produced by Direct-Dilution
are Comparable in Size, and PDI.

| Method | Size (nm) | PDI |
|---|---|---|
| Dialysis | 119 +/− 7 | 0.05 +/− 0.01 |
| Direct Dilution | 145 +/− 17 | 0.25 +/− 0.09 |

Values are the mean +/− STDEV.
Dialysis N = 6,
Direct Dilution N = 11

TABLE 3
Direct-Dilution Formulation Reduces the Preparation
Time and increases the Yield and Potency of saRNA-
LNPs during the Vaccine Manufacture.

| Method | Time Required (min) | Yield Percent |
|---|---|---|
| Dialysis | 330* | 64 +/− 2 |
| Direct Dilution | 15* | 91 +/− 7 |

*Estimated post-formulation processing times. Yield values are the mean +/− STDEV. Dialysis N = 5, Direct Dilution N = 7. Yield calculated as total resulting encapsulated RNA divided by total RNA input.

TABLE 4
Direct-Dilution Formulation yields saRNA-LNPs that are Stable to Storage.

| | | Storage | | | | | | | | | |
|---|---|---|---|---|---|---|---|---|---|---|---|
| Method | Buffer | Temp C. | 3 D | 7 D | 14 D | 21 D | 28 D | 56 D | 6 M | 9 M | 12 M |
| Direct Dilution | 1 | −80 | 13.5 | 20.2 | 37.8 | 32.7 | 18.4 | 8.7 | 5 | 16.8 | 4.9 |
| Dialysis | 1 | −80 | 5.2 | 1.9 | 16.5 | 9.6 | 9.2 | 2 | 1.8 | 4.6 | 1 |
| Direct Dilution | 1 | −20 | 7.7 | 6.6 | 7.4 | 13.9 | 6.4 | 1.7 | 0.2 | 0.7 | 0.1 |
| Dialysis | 1 | −20 | 1.1 | 1.4 | 5.2 | 5.6 | 2.9 | 1.3 | 0.1 | 0.4 | 0.1 |
| Direct Dilution | 1 | 4 | 10.5 | 22 | 32 | 20.9 | 22.5 | 8.1 | 0.1 | | |
| Dialysis | 1 | 4 | 6.5 | 1.1 | 3.6 | 2.6 | 1.9 | 0.4 | 0.1 | | |
| Direct Dilution | 1 | 22 | 13.7 | 14.2 | 15 | 2 | 4.3 | 0.5 | 0.1 | | |
| Dialysis | 1 | 22 | 0.6 | 0.6 | 1.4 | 2.3 | 0.7 | 0.4 | 0.1 | | |
| Direct Dilution | 2 | −80 | 20.4 | 46.2 | 56.6 | 65.7 | 53.3 | 40.2 | 33.5 | 49.9 | 28.3 |
| Dialysis | 2 | −80 | 15.3 | 4.5 | 25.1 | 26.5 | 21.5 | 12.4 | 6.3 | 12.5 | 3.6 |
| Direct Dilution | 2 | −20 | 17.9 | 21.5 | 9.2 | 13.1 | 4.7 | 0.6 | 0.1 | 0.4 | 0.1 |
| Dialysis | 2 | −20 | 2.3 | 2.5 | 4.2 | 5.4 | 2.2 | 1 | 0.2 | 0.3 | 0.1 |
| Direct Dilution | 2 | 4 | 19 | 25.3 | 29.2 | 22.1 | 10.6 | 1.6 | 0.1 | | |
| Dialysis | 2 | 4 | 2.6 | 2.1 | 6.2 | 3.5 | 2.5 | 0.5 | 0.2 | | |
| Direct Dilution | 2 | 22 | 15.6 | 8.2 | 3.9 | 0.9 | 0.4 | 0.3 | 0.1 | | |
| Dialysis | 2 | 22 | 1.9 | 1.1 | 0.7 | 0.8 | 0.4 | 0.4 | 0.2 | | |

Vaccines prepared using proprietary lipid and stored at 0.5 ug/ml. Values are the percentage of cells in the 488 (GFP) positive gate of transfected BHK cells.
D = Days,
M = Months

In one embodiment the combining of the two liquids is by mixing. For example, the mixing is by microfluidic mixing by chaotic advection. In another embodiment, T-junction mixing can be used at larger scales resulting in similar LNPs.

Microfluidic devices provide an ability to controllably and rapidly mix fluids at the nanoliter scale with precise control over temperature, residence times, and solute concentrations. Controlled and rapid microfluidic mixing has been previously applied in the synthesis of inorganic nanoparticles and microparticles and can outperform macroscale systems in large scale production of nanoparticles. Microfluidic two-phase droplet techniques have been applied to produce monodisperse polymeric microparticles for drug delivery or to produce large vesicles for the encapsulation of cells, proteins, or other biomolecules. In some embodiments, the use of hydrodynamic flow focusing, a common microfluidic technique to provide rapid mixing of reagents, to create monodisperse liposomes of controlled size is used. This technique has also proven useful in the production of polymeric nanoparticles where smaller, more monodisperse particles were obtained, with higher encapsulation of small molecules as compared to bulk production methods.

In one embodiment, at least or about 1, 2, 3, 4, 5, 6, 7, 8, 9, 10, 11, 12, 13, 14, 15, or more lipids are solubilized in an organic solvent such as ethanol at a concentration below 350 mM, while the nucleic acid is in a pH 3-6, preferably an acetic acid buffer. These two streams meet in a common microfluidic channel and are forced to mix in several milliseconds before being ejected into an aqueous recipient well. Two events occur while mixing: 1) the initially neutral ionizable lipid contacts the low pH buffer and becomes protonated at the same time as mixing with the anionic saRNA thus forming electrostatic bonds between the cationic lipid and anionic nucleic acid; and 2) the lipids become insoluble in the predominantly aqueous buffer and encapsulate the mRNA. The pH in the final well containing PBS is typically 6-6.5 due to the mixture of acetic acid, PBS and ionizable lipid, all of which are buffers with a certain buffer capacity and initial pH.

In some embodiments, the LNPs are dialyzed against PBS to raise the pH to about 7.4 and remove ethanol. LNP assembly continues during dialysis as the ionizable lipid with an LNP pKa near 6.5 becomes gradually neutralized to pH 7.4 and thereby less soluble triggering a fusion of the LNPs that increases size and transforms an aqueous electron lucent core to an electron dense core containing predominantly the ionizable lipid and nucleic acid.

The total amount of lipid provided by the invention in the composition being administered is, in one embodiment, from about 2 to about 100 mg lipid per mg biologically active agent (e.g., saRNA), in another embodiment from about 5 to about 25 mg lipid per mg biologically active agent (e.g., saRNA), in another embodiment from about 7 to about 25 mg lipid per mg biologically active agent (e.g., saRNA) and in one embodiment from about 10 to about 20 mg lipid per mg biologically active agent (e.g., saRNA).

### Pharmaceutical Compositions and Methods

Following dilution, the LNPs encapsulating saRNA of the present invention may be used to deliver a therapeutic or prophylactic agent to a cell, in vitro or in vivo. In particular embodiments, the therapeutic agent is a nucleic acid, which is delivered to a cell using nucleic acid-lipid particles of the present invention. The methods and compositions may be readily adapted for the delivery of any suitable therapeutic agent for the treatment of any disease or disorder that would benefit from such treatment.

In certain embodiments, the present invention provides methods for introducing a nucleic acid into a cell. Preferred nucleic acids for introduction into cells are mRNA, saRNA, miRNA, immune-stimulating oligonucleotides, DNA plasmids, antisense and ribozymes. These methods may be carried out by contacting the particles or compositions of the present invention with the cells for a period of time sufficient for intracellular delivery to occur.

Nucleic acids for use with this invention may be prepared according to any available technique. For mRNA, the primary methodology of preparation is, but not limited to, enzymatic synthesis (also termed in vitro transcription) which currently represents the most efficient method to produce long sequence-specific mRNA. In vitro transcription describes a process of template-directed synthesis of RNA molecules from an engineered DNA template comprised of an upstream bacteriophage promoter sequence (e.g. including but not limited to that from the T7, T3 and SP6 coliphage) linked to a downstream sequence encoding the gene of interest. Template DNA can be prepared for in vitro transcription from a number of sources with appropriate techniques which are well known in the art including, but not limited to, plasmid DNA and polymerase chain reaction amplification (see Linpinsel, J. L and Conn, G. L., General protocols for preparation of plasmid DNA template and Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N.Y. Humana Press, 2012)

Transcription of the RNA occurs in vitro using the linearized DNA template in the presence of the corresponding RNA polymerase and adenosine, guanosine, uridine and cytidine ribonucleoside triphosphates (rNTPs) under conditions that support polymerase activity while minimizing potential degradation of the resultant mRNA transcripts. In vitro transcription can be performed using a variety of commercially available kits including, but not limited to RiboMax Large Scale RNA Production System (Promega), MegaScript Transcription kits (Life Technologies) as well as with commercially available reagents including RNA polymerases and rNTPs. The methodology for in vitro transcription of mRNA is well known in the art. (see, e.g. Losick, R., 1972, In vitro transcription, Ann Rev Biochem v.41 409-46; Kamakaka, R. T. and Kraus, W. L. 2001. In Vitro Transcription. Current Protocols in Cell Biology. 2:11.6:11.6.1-11.6.17; Beckert, B. And Masquida, B., (2010) Synthesis of RNA by In Vitro Transcription in RNA in Methods in Molecular Biology v. 703 (Neilson, H. Ed), New York, N.Y. Humana Press, 2010; Brunelle, J. L. and Green, R., 2013, Chapter Five—In vitro transcription from plasmid or PCR-amplified DNA, Methods in Enzymology v. 530, 101-114; all of which are incorporated herein by reference).

The desired in vitro transcribed saRNA is then purified from the undesired components of the transcription or associated reactions (including unincorporated rNTPs, protein enzyme, salts, short RNA oligos etc.). Techniques for the isolation of the saRNA transcripts are well known in the art. Well known procedures include phenol/chloroform extraction or precipitation with either alcohol (ethanol, isopropanol) in the presence of monovalent cations or lithium chloride. Additional, non-limiting examples of purification procedures which can be used include size exclusion chromatography (Lukavsky, P. J. and Puglisi, J. D., 2004, Large-scale preparation and purification of polyacrylamide-free RNA oligonucleotides, RNA v.10, 889-893), silica-based affinity chromatography and polyacrylamide gel electrophoresis (Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N.Y. Humana Press, 2012). Purification can be performed using a variety of commercially available kits including, but not limited to SV Total Isolation System (Promega) and In Vitro Transcription Cleanup and Concentration Kit (Norgen Biotek).

In a further aspect, the invention provides a pharmaceutical composition comprising a lipid particle of the invention and a pharmaceutically acceptable carrier or diluent. Representative pharmaceutically acceptable carriers or diluents include solutions for intravenous injection (e.g., saline or dextrose). The composition can take the form of a cream, ointment, gel, suspension, or emulsion.

As used herein, “treatment” includes ameliorative, curative and prophylactic treatment. As used herein, a “patient” means an animal, preferably a mammal, preferably a human, in need of treatment.

The term “therapeutically effective amount” refers to the amount of the compound of the invention and the biologically active agent (e.g. the therapeutic compound) needed to treat or ameliorate a targeted disease or condition.

The term “immunologically effective amount” refers to the amount of the compound of the invention and of RNA which encodes an immunogen needed to elicit an immune response which recognizes the immunogen (e.g. in the context of a pathogen). The term “immunogen” refers to any substance or organism that provokes an immune response when introduced into the body. The phrase “RNA which encodes an immunogen” refers to a polynucleotide, such as a messenger RNA or a replicon (e.g., self-replicating RNA), that when administered to a cell or organism is capable of being translated into a polypeptide according to the codon sequence of such RNA.

By “proliferative disease” as used herein is meant any disease, condition, trait, genotype or phenotype characterized by unregulated cell growth or replication as is known in the art. In one embodiment, the proliferative disease is cancer. In one embodiment, the proliferative disease is a tumor. In one embodiment, the proliferative disease includes, but are not limited to, e.g., liquid tumors such as, e.g., leukemias, e.g., acute myelogenous leukemia (AML), chronic myelogenous leukemia (CML), acute lymphocytic leukemia (ALL), multiple myeloma, and chronic lymphocytic leukemia; and solid tumors, e.g., AIDS related cancers such as Kaposi's sarcoma; breast cancers; bone cancers; brain cancers; cancers of the head and neck, non-Hodgkins lymphoma, adenoma, squamous cell carcinoma, laryngeal carcinoma, gallbladder and bile duct cancers, cancers of the retina, cancers of the esophagus, gastrointestinal cancers, ovarian cancer, uterine cancer, thyroid cancer, testicular cancer, endometrial cancer, melanoma, colorectal cancer, lung cancer, bladder cancer, prostate cancer, lung cancer (including non-small cell lung carcinoma), pancreatic cancer, sarcomas, Wilms' tumor, cervical cancer, head and neck cancer, skin cancers, nasopharyngeal carcinoma, liposarcoma, epithelial carcinoma, renal cell carcinoma, gallbladder adeno carcinoma, endometrial sarcoma, multidrug resistant cancers. In one embodiment, the proliferative disease includes neovascularization associated with tumor angiogenesis, macular degeneration (e.g. wet/dry age-related macular degeneration), corneal neovascularization, diabetic retinopathy, neovascular glaucoma, myopic degeneration. In one embodiment, the proliferative disease includes restenosis and polycystic kidney disease.

By “autoimmune disease” as used herein is meant any disease, condition, trait, genotype or phenotype characterized by autoimmunity as is known in the art. Autoimmune diseases include, but are not limited to, e.g., multiple sclerosis, diabetes mellitus, lupus, scleroderms, fibromyalgia, transplantation rejection (e.g. prevention of allograft rejection), pernicious anemia, rheumatoid arthritis, systemic lupus erythematosus, dermatomyositis, myasthenia gravis, lupus erythematosus, multiple sclerosis, and Grave's disease.

By “infectious disease” is meant any disease, disorder or condition associated with an infectious agent, such as a virus, bacteria, fungus, prion or parasite. The invention can be used to actively or passively immunize against pathogens which cause infectious disease. Examples of such pathogens are given below.

By “neurologic disease” is meant any disease, disorder, or condition affecting the central or peripheral nervous system. Neurologic diseases include, but are not limited to, diseases or disorders of either the peripheral or the central nervous system including, e.g., Alzheimer's Disease, Aneurysm, Brain Injury, Carpal Tunnel Syndrome, Cerebral Aneurysm, Chronic Pain, Creutzfeldt-Jakob Disease, Epilepsy, Huntington's Disease, Meningitis, Seizure Disorders, and other neurologic diseases, disorders and syndromes.

By “respiratory disease” is meant any disease or condition affecting the respiratory tract. Respiratory diseases include, but are not limited to, e.g., asthma, chronic obstructive pulmonary disease (COPD), allergic rhinitis, sinusitis, allergies, impeded respiration, respiratory distress syndrome, cystic fibrosis, pulmonary hypertension or vasoconstriction and emphysema.

By “cardiovascular disease” is meant and disease or condition affecting the heart and vasculature. Cardiovascular diseases include, but are not limited to, e.g., coronary heart disease (CHD), cerebrovascular disease (CVD), aortic stenosis, peripheral vascular disease, myocardial infarction (heart attack), arrhythmia, ischemia, and congestive heart failure.

By “ocular disease” as used herein is meant any disease, condition, trait, genotype or phenotype of the eye and related structures. Ocular diseases include, but are not limited to, e.g., cystoid macular edema, diabetic retinopathy, lattice degeneration, retinal vein occlusion, retinal artery occlusion, macular degeneration (e.g., age related macular degeneration such as wet AMD or dry AMD), toxoplasmosis, retinitis pigmentosa, conjunctival laceration, corneal laceration, glaucoma, and the like.

By “metabolic disease” is meant any disease or condition affecting metabolic pathways. Metabolic disease can result in an abnormal metabolic process, either congenital due to inherited enzyme abnormality (inborn errors of metabolism) or acquired due to disease of an endocrine organ or failure of a metabolically important organ such as the liver. In one embodiment, metabolic disease includes obesity, insulin resistance, and diabetes (e.g. type I and/or type II diabetes).

By “dermatological disease” is meant any disease or condition of the skin, dermis, or any substructure therein such as a hair, a follicle, etc. Dermatological diseases, disorders, conditions, and traits can include psoriasis, ectopic dermatitis, skin cancers such as melanoma and basal cell carcinoma, hair loss, hair removal and alterations in pigmentation.

By “auditory disease” is meant any disease or condition of the auditory system, including the ear, such as the inner ear, middle ear, outer ear, auditory nerve, and any substructures therein. Auditory diseases, disorders, conditions, and traits can include hearing loss, deafness, tinnitus, vertigo, balance and motion disorders.

By “regenerative disease” is meant any disease or condition where insufficient cell or tissue generation or regeneration in vivo or in vitro prevents the establishment or restoration of proper organ function before or after injury, prevents or slows wound healing or resolution of ulcerative lesions, accelerates ageing, or prevents effective cell-based therapy. The term “messenger ribonucleic acid” (messenger RNA, mRNA) refers to a ribonucleic acid (RNA) molecule that mediates the transfer of genetic information to ribosomes in the cytoplasm, where it serves as a template for protein synthesis. It is synthesized from a DNA template during the process of transcription. See, The American Heritage®. Dictionary of the English Language, Fourth Edition (Updated in 2009). Houghton Mifflin Company.

In eukaryotes, mRNA is transcribed in vivo at the chromosomes by the cellular enzyme RNA polymerase. During or after transcription in vivo, a 5′ cap (also termed an RNA cap, an RNA 7-methylguanosine cap, or an RNA m7G cap) is added in vivo to the 5′ end of the mRNA. The 5′ cap is terminal 7-methylguanosine residue that is linked through a 5′-5′-triphosphate bond to the first transcribed nucleotide. In addition, most eukaryotic mRNA molecules have a polyadenylyl moiety (“poly(A) tail”) at the 3′ end of the mRNA molecule. In vivo, the eukaryotic cell adds the poly(A) tail after transcription, often at a length of about 250 adenosine residues (SEQ ID NO: 12). Thus, a typical mature eukaryotic mRNA has a structure that begins at the 5′ end with an mRNA cap nucleotide followed by a 5′ untranslated region (5′UTR) of nucleotides, then an open reading frame that begins with a start codon which is an AUG triplet of nucleotide bases, that is the coding sequence for a protein, and that ends with a stop codon that may be a UAA, UAG, or UGA triplet of nucleotide bases, then a 3′ untranslated region (3′UTR) of nucleotides and ending with a poly-adenosine tail. While the features of the typical mature eukaryotic mRNA are made naturally in a eukaryotic cell in vivo, the same or structurally and functionally equivalent features can be made in vitro using the methods of molecular biology. Accordingly, any RNA having the structure similar to a typical mature eukaryotic mRNA can function as a mRNA and is within the scope of the term “messenger ribonucleic acid”.

The saRNA molecule is generally of a size that it can be encapsulated in a lipid nanoparticle of the invention. While the size of a saRNA molecule varies in nature depending upon the identity of the saRNA species that encodes for a particular protein, an average size for a saRNA molecule is average saRNA size is 500-10,000 bases.

### Pharmaceutical Formulation of LNP Compositions

For pharmaceutical use, the LNP compositions of the invention may be administered by enteral or parenteral routes, including intravenous, intramuscular, subcutaneous, transdermal, airway (aerosol), oral, intranasal, rectal, vaginal, buccal, nasopharyngeal, gastrointestinal or sublingual administration. The administration may be systemic (e.g., IV) or local (e.g., IM, SC, TD, intranasal, or topical). Topical administration may involve, e.g., catheterization, implantation, osmotic pumping, direct injection, dermal/transdermal application, stenting, ear/eye drops or portal vein administration. The compounds of formula (I) should be assessed for their biopharmaceutical properties, such as solubility and solution stability (across pH), permeability, etc., in order to select the most appropriate dosage form and route of administration for treatment of the proposed indication.

The compositions of the invention will generally, but not necessarily, be administered as a formulation in association with one or more pharmaceutically acceptable excipients. The term “excipient” includes any ingredient other than the compound(s) of the invention, the other lipid component(s) and the biologically active agent. An excipient may impart either a functional (e.g. drug release rate controlling) and/or a non-functional (e.g. processing aid or diluent) characteristic to the formulations. The choice of excipient will to a large extent depend on factors such as the particular mode of administration, the effect of the excipient on solubility and stability, and the nature of the dosage form.

Typical pharmaceutically acceptable excipients include: diluents, e.g. lactose, dextrose, sucrose, mannitol, sorbitol, cellulose and/or glycine; lubricants, e.g. silica, talcum, stearic acid, its magnesium or calcium salt and/or polyethyleneglycol; binders, e.g. magnesium aluminum silicate, starch paste, gelatin, tragacanth, methylcellulose, sodium carboxymethylcellulose and/or polyvinyl pyrrolidone; disintegrants, e.g. starches, agar, alginic acid or its sodium salt, or effervescent mixtures; and/or absorbents, colorants, flavors and/or sweeteners.

In some embodiments, the LNPs stored frozen and thawed before use. In some embodiments, that are stable at 4° C. for up to 2 weeks. In some embodiments, the LNPs are in various cryopreservation solutions containing various sugars for freezing that remain at the time of injection.

The buffer may be an aqueous solution carrier which may optionally contain a buffer (e.g. a cryobuffer) and/or a sugar.

A thorough discussion of pharmaceutically acceptable excipients is available in Gennaro, Remington: The Science and Practice of Pharmacy 2000, 20th edition (ISBN: 0683306472).

The compositions of the invention may be administered orally. Oral administration may involve swallowing, so that the compound enters the gastrointestinal tract, and/or buccal, lingual, or sublingual administration by which the compound enters the blood stream directly from the mouth.

The compositions of the invention can be administered parenterally. The compounds and compositions of the invention may be administered directly into the blood stream, into subcutaneous tissue, into muscle, or into an internal organ. Suitable means for administration include intravenous, intraarterial, intrathecal, intraventricular, intraurethral, intrasternal, intracranial, intramuscular, intrasynovial and subcutaneous. Suitable devices for administration include needle (including microneedle) injectors, needle-free injectors and infusion techniques.

Parenteral formulations are typically aqueous or oily solutions. Where the solution is aqueous, excipients such as sugars (including but not restricted to glucose, mannitol, sorbitol, etc.) salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water (WFI).

Parenteral formulations may include implants derived from degradable polymers such as polyesters (i.e. polylactic acid, polylactide, polylactide-co-glycolide, polycapro-lactone, polyhydroxybutyrate), polyorthoesters and polyanhydrides. These formulations may be administered via surgical incision into the subcutaneous tissue, muscular tissue or directly into specific organs.

The preparation of parenteral formulations under sterile conditions, e.g., by lyophilization, may readily be accomplished using standard pharmaceutical techniques well known to the skilled person.

The solubility of the compounds and compositions used in the preparation of parenteral solutions may be increased by the use of appropriate formulation techniques, such as the incorporation of co-solvents and/or solubility-enhancing agents such as surfactants, micelle structures and cyclodextrins.

The compositions of the invention can be administered intranasally or by inhalation, typically in the form of a dry powder (either alone, as a mixture, e.g., in a dry blend with lactose, or as a mixed component particle, e.g., mixed with phospholipids, such as phosphatidylcholine) from a dry powder inhaler, as an aerosol spray from a pressurized container, pump, spray, atomizer (preferably an atomizer using electrohydrodynamic to produce a fine mist), or nebulizer, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, e.g., chitosan or cyclodextrin.

The pressurized container, pump, spray, atomizer, or nebulizer contains a solution or suspension of the compound(s) of the invention comprising, e.g., ethanol, aqueous ethanol, or a suitable alternative agent for dispersing, solubilizing, or extending release of the compositions of the invention, a propellant(s) as solvent and an optional surfactant, such as sorbitan trioleate, oleic acid, or an oligolactic acid.

Prior to use in a dry powder or suspension formulation, the composition is micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying.

Capsules (made, e.g., from gelatin or hydroxypropylmethylcellulose), blisters and cartridges for use in an inhaler or insufflator may be formulated to contain a powder mix of the compound or composition of the invention, a suitable powder base such as lactose or starch and a performance modifier such as I-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

Formulations for inhaled/intranasal administration may be formulated to be immediate and/or modified release using, e.g., PGLA. Modified release formulations include delayed-, sustained-, pulsed-, controlled-, targeted and programmed release.

Suitable formulations for transdermal application include a therapeutically effective amount of a compound or composition of the invention with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

Lipid compositions of the invention are administered in any of a number of ways, including parenteral, intravenous, systemic, local, oral, intratumoral, intramuscular, subcutaneous, intraperitoneal, inhalation, or any such method of delivery. In one embodiment, the compositions are administered parenterally, i.e., intraarticularly, intravenously, intraperitoneally, subcutaneously, or intramuscularly. In a specific embodiment, the liposomal compositions are administered by intravenous infusion or intraperitoneally by a bolus injection.

Lipid compositions of the invention can be formulated as pharmaceutical compositions suitable for delivery to a subject. The pharmaceutical compositions of the invention will often further comprise one or more buffers (e.g., neutral buffered saline or phosphate buffered saline), carbohydrates (e.g., glucose, mannose, sucrose, dextrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate.

Suitable formulations for use in the present invention can be found, e.g., in Remington's Pharmaceutical Sciences, Mack Publishing Company, Philadelphia, Pa., 17.sup.th Ed. (1985). Often, compositions will comprise a solution of the lipid nanoparticles suspended in an acceptable carrier, such as an aqueous carrier.

In one embodiment, this invention provides for a pharmaceutical composition (i.e. formulation) comprising a lipid composition of the invention and a pharmaceutically acceptable carrier or excipient. In another embodiment at least one other lipid component is present in the lipid composition. In another embodiment the lipid composition is in the form of a liposome. In another embodiment the lipid composition is in the form of a lipid nanoparticle. In another embodiment the lipid composition is suitable for delivery to the liver. In another embodiment the lipid composition is suitable for delivery to a tumor. In another embodiment the lipid composition is suitable for local delivery applications (eye, ear, skin, lung); delivery to muscle (i.m.), fat, or subcutaneous cells (s.c. dosing). In another embodiment the biologically active agent is a RNA or DNA.

For immunization purposes a composition will generally be prepared as an injectable and will be administered by injection (e.g. by intramuscular injection).

The invention also provides a delivery device (e.g. syringe, nebulizer, sprayer, inhaler, dermal patch, etc.) containing a composition of the invention. This device can be used to administer a pharmaceutical composition to a subject e.g. to a human for immunization.

### Cells and Organs Targeted by the Pharmaceutical Compositions

The compositions, methods and uses of the invention can be used to deliver a biologically active agent to one or more of the following in a patient: the liver or liver cells (e.g. hepatocytes); a kidney or kidney cells; a tumor or tumor cells; the CNS or CNS cells (Central Nervous System, e.g. brain and/or spinal cord); the PNS or PNS cells (Peripheral Nervous System); a lung or lung cells; the vasculature or vascular cells; the skin or skin cells (e.g. dermis cells and/or follicular cells); an eye or ocular cells (e.g. macula, fovea, cornea, retina), and an ear or cells of the ear (e.g. cells of the inner ear, middle ear and/or outer ear).

The compositions, methods and uses of the invention can also be used to deliver a biologically active agent (e.g. RNA which encodes an immunogen) to cells of the immune system.

In one embodiment, the compositions, methods and uses of the invention are for delivering a biologically active agent to liver cells (e.g. hepatocytes). In one embodiment, the compounds, compositions, methods and uses of the invention are for delivering a biologically active agent to a tumor or to tumor cells (e.g. a primary tumor or metastatic cancer cells). In another embodiment, the compounds, compositions, methods and uses are for delivering a biologically active agent to the skin adipose, muscle and lymph nodes (i.e. sc dosing).

For delivery of a biologically active agent to the liver or liver cells, in one embodiment a composition of the invention is contacted with the liver or liver cells of the patient as is generally known in the art, such as via parental administration (e.g. intravenous, intramuscular, subcutaneous administration) or local administration (e.g. direct injection, portal vein injection, catheterization, stenting), to facilitate delivery.

For delivery of a biologically active agent to the kidney or kidney cells, in one embodiment a composition of the invention is contacted with the kidney or kidney cells of the patient as is generally known in the art, such as via parental administration (e.g. intravenous, intramuscular, subcutaneous administration) or local administration (e.g. direct injection, catheterization, stenting), to facilitate delivery.

For delivery of a biologically active agent to a tumor or tumor cells, in one embodiment a composition of the invention is contacted with the tumor or tumor cells of the patient as is generally known in the art, such as via parental administration (e.g. intravenous, intramuscular, subcutaneous administration) or local administration (e.g. direct injection, catheterization, stenting), to facilitate delivery.

For delivery of a biologically active agent to the CNS or CNS cells (e.g. brain cells and/or spinal cord cells), in one embodiment a composition of the invention is contacted with the CNS or CNS cells (e.g. brain cells and/or spinal cord cells) of the patient as is generally known in the art, such as via parental administration (e.g. intravenous, intramuscular, subcutaneous administration) or local administration (e.g. direct injection, catheterization, stenting, osmotic pump administration (e.g. intrathecal or ventricular)), to facilitate delivery.

For delivery of a biologically active agent to the PNS or PNS cells, in one embodiment a composition of the invention is contacted with the PNS or PNS cells of the patient as is generally known in the art, such as via parental administration (e.g. intravenous, intramuscular, subcutaneous administration) or local administration (e.g. direct injection), to facilitate delivery.

For delivery of a biologically active agent to a lung or lung cells, in one embodiment a composition of the invention is contacted with the lung or lung cells of the patient as is generally known in the art, such as via parental administration (e.g. intravenous, intramuscular, subcutaneous administration) or local administration (e.g. pulmonary administration directly to lung tissues and cells), to facilitate delivery.

For delivery of a biologically active agent to the vasculature or vascular cells, in one embodiment a composition of the invention is contacted with the vasculature or vascular cells of the patient as is generally known in the art, such as via parental administration (e.g. intravenous, intramuscular, subcutaneous administration) or local administration (e.g. clamping, catheterization, stenting), to facilitate delivery.

For delivery of a biologically active agent to the skin or skin cells (e.g. dermis cells and/or follicular cells), in one embodiment a composition of the invention is contacted with the skin or skin cells (e.g. dermis cells and/or follicular cells) of the patient as is generally known in the art, such as via parental administration (e.g. intravenous, intramuscular, subcutaneous administration) or local administration (e.g. direct dermal application, iontophoresis), to facilitate delivery.

For delivery of a biologically active agent to an eye or ocular cells (e.g. macula, fovea, cornea, retina), in one embodiment a composition of the invention is contacted with the eye or ocular cells (e.g. macula, fovea, cornea, retina) of the patient as is generally known in the art, such as via parental administration (e.g. intravenous, intramuscular, subcutaneous administration) or local administration (e.g. direct injection, intraocular injection, periocular injection, subretinal, iontophoresis, use of eyedrops, implants), to facilitate delivery.

For delivery of a biologically active agent to an ear or cells of the ear (e.g. cells of the inner ear, middle ear and/or outer ear), in one embodiment composition of the invention is contacted with the ear or cells of the ear (e.g. cells of the inner ear, middle ear and/or outer ear) of the patient as is generally known in the art, such as via parental administration (e.g. intravenous, intramuscular, subcutaneous administration) or local administration (e.g. direct injection), to facilitate delivery.

For delivery of a biologically active agent (e.g. saRNA encoding an immunogen) to cells of the immune system (e.g. antigen-presenting cells, including professional antigen presenting cells), in one embodiment composition of the invention is delivered intramuscularly, after which immune cells can infiltrate the delivery site and process delivered RNA. Such immune cells can include macrophages (e.g. bone marrow derived macrophages), dendritic cells (e.g. bone marrow derived plasmacytoid dendritic cells and/or bone marrow derived myeloid dendritic cells), monocytes (e.g. human peripheral blood monocytes), etc. (e.g. see WO2012/006372).

### Immunization According to the Invention

For immunization purposes, in some embodiments, the invention encompasses delivering a saRNA that encodes an immunogen. The immunogen elicits an immune response which recognizes the immunogen and so can be used to provide immunity against a pathogen, or against an allergen, or against a tumor antigen. Immunizing against disease and/or infection caused by a pathogen is preferred.

In certain embodiments the LNPs have adjuvant characteristics. For example, the LNPs disclosed herein can have specific T follicular helper cell adjuvant activity that leading to potent antibody responses. An asymmetric ionizable lipid LNP may act as a strong Th2-biased adjuvant when delivered with protein subunit antigens. In some embodiments, LNP mRNA vaccines disclosed herein drive a Tfh-biased response that stimulates the proliferation of Tfh and germinal center B cells and a potent long-lived neutralizing antibody response.

The RNA is delivered with a lipid composition of the invention (e.g. formulated as an LNP). In some embodiments, the invention utilizes liposomes within which immunogen-encoding RNA is encapsulated. Encapsulation within LNPs can protect RNA from RNase digestion. The encapsulation efficiency does not have to be 100%. Presence of external RNA molecules (e.g. on the exterior surface of liposome) or “naked” RNA molecules (RNA molecules not associated with an LNP) is acceptable. Preferably, for a composition comprising liposomes and RNA molecules, at least half of the RNA molecules (e.g., at least 50%, at least 55%, at least 60%, at least 65%, at least 70%, at least 75%, at least 80%, at least 85%, at least 90%, or at least 95% of the RNA molecules) are encapsulated in the LNPs.

RNA molecules may also be complexed with LNPs. For example, it is not necessary that the lipid forms liposomes (with aqueous core) only. Some lipid nanoparticles may comprise a lipid core (e.g., the composition may comprise a mixture of liposomes and nanoparticles with a lipid core). In such cases, the RNA molecules may be encapsulated by LNPs that have an aqueous core, and complexed with the LNPs that have a lipid core by non-covalent interactions (e.g., ionic interactions between negatively charged RNA and cationic lipid). Encapsulation and complexation with LNPs (whether with a lipid or aqueous core) can protect RNA from RNase digestion. The encapsulation/complexation efficiency does not have to be 100%. Presence of “naked” RNA molecules (RNA molecules not associated with a liposome) is acceptable. Preferably, for a composition comprising a population of LNPs and a population of RNA molecules, at least half of the population of RNA molecules (e.g., at least 50%, at least 55%, at least 60%, at least 65%, at least 70%, at least 75%, at least 80%, at least 85%, at least 90%, or at least 95% of the RNA molecules) are either encapsulated in LNPs, or complexed with LNPs.

### RNA Molecules in the Pharmaceutical Compositions

After in vivo administration of an immunization composition, the delivered RNA is released and is translated inside a cell to provide the immunogen in situ. In certain embodiments, the RNA is plus (“+”) stranded, so it can be translated by cells without needing any intervening replication steps such as reverse transcription. It may also bind to TLR7 receptors expressed by immune cells, thereby initiating an adjuvant effect. Additionally, or alternatively, the RNA may bind other receptors such as RIG I, MDA5, or RIG I and MDA5.

In certain embodiments, the RNA is a self-replicating RNA. A self-replicating RNA molecule (replicon) can, when delivered to a vertebrate cell even without any proteins, lead to the production of multiple daughter RNAs by transcription from itself (via an antisense copy which it generates from itself). A self-replicating RNA molecule is thus, in certain embodiments, a (+) strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded immunogen, or may be transcribed to provide further transcripts with the same sense as the delivered RNA which are translated to provide in situ expression of the immunogen. The overall result of this sequence of transcriptions is a huge amplification in the number of the introduced replicon RNAs and so the encoded immunogen becomes a major polypeptide product of the host cells.

One suitable system for achieving self-replication is to use an alphavirus-based RNA replicon. These (+) stranded replicons are translated after delivery to a cell to give of a replicase (or replicase-transcriptase). The replicase is translated as a polyprotein which auto cleaves to provide a replication complex which creates genomic (−) strand copies of the (+) strand delivered RNA. These (−) strand transcripts can themselves be transcribed to give further copies of the +stranded parent RNA and also to give a subgenomic transcript which encodes the immunogen. Translation of the subgenomic transcript thus leads to in situ expression of the immunogen by the infected cell. Suitable alphavirus replicons can use a replicase from a Sindhis virus, a Semliki forest virus, an eastern equine encephalitis virus, a Venezuelan equine encephalitis virus, etc. Mutant or wild-type viruses' sequences can be used e.g. the attenuated TC83 mutant of VEEV has been used in replicons.

A preferred self-replicating RNA molecule thus encodes (i) a RNA-dependent RNA polymerase which can transcribe RNA from the self-replicating RNA molecule and (ii) an immunogen. The polymerase can be an alphavirus replicase e.g. comprising one or more of alphavirus proteins nsP1, nsP2, nsP3 and nsP4.

Whereas natural alphavirus genomes encode structural virion proteins in addition to the nonstructural replicase polyprotein, in particular embodiments, a self-replicating RNA molecule of the invention does not encode alphavirus structural proteins. Thus, a particular self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the invention and their place is taken by gene(s) encoding the immunogen of interest, such that the subgenomic transcript encodes the immunogen rather than the structural alphavirus virion proteins.

Thus, a self-replicating RNA molecule useful with the invention may have two open reading frames. One open reading frame encodes a replicase, e.g., the first, (5′) open reading frame; the other open reading frame encodes an immunogen, e.g., the second, (3′) open reading frame. In some embodiments the RNA may have additional (e.g. downstream) open reading frames e.g. to encode further immunogens (see below) or to encode accessory polypeptides.

A self-replicating RNA molecule can have a 5′ sequence which is compatible with the encoded replicase.

Self-replicating RNA molecules can have various lengths, but they are typically 5000-25000 nucleotides long e.g. 8000-15000 nucleotides, or 9000-12000 nucleotides. Thus, the RNA is longer than seen in siRNA or conventional mRNA delivery. In some embodiments, the self-replicating RNA is greater than about 2000 nucleotides, such as greater than about: 9000, 12000, 15000, 18000, 21000, 24000, or more nucleotides long

An RNA molecule may have a 5′ cap (e.g. a 7-methylguanosine). This cap can enhance in vivo translation of the RNA.

The 5′ nucleotide of a RNA molecule useful with the invention may have a 5′ triphosphate group. In a capped RNA this may be linked to a 7-methylguanosine via a 5′-to-5′ bridge. A 5′ triphosphate can enhance RIG-I binding and thus promote adjuvant effects.

An RNA molecule may have a 3′ poly A tail. It may also include a poly A polymerase recognition sequence (e.g. AAUAAA) near its 3′ end.

An RNA molecule useful with the invention for immunization purposes will typically be single-stranded. Single-stranded RNAs can generally initiate an adjuvant effect by binding to TLR7, TLR8, RNA helicases and/or PKR. RNA delivered in double-stranded form (dsRNA) can bind to TLR3, and this receptor can also be triggered by dsRNA which is formed either during replication of a single-stranded RNA or within the secondary structure of a single-stranded RNA.

RNA molecules for immunization purposes can conveniently be prepared by in vitro transcription (IVT). IVT can use a (cDNA) template created and propagated in plasmid form in bacteria, or created synthetically (for example by gene synthesis and/or polymerase chain-reaction (PCR) engineering methods). For instance, a DNA-dependent RNA polymerase (such as the bacteriophage T7, T3 or SP6 RNA polymerases) can be used to transcribe the RNA from a DNA template. Appropriate capping and poly A addition reactions can be used as required (although the replicon's poly-A is usually encoded within the DNA template). These RNA polymerases can have stringent requirements for the transcribed 5′ nucleotide(s) and in some embodiments these requirements must be matched with the requirements of the encoded replicase, to ensure that the IVT transcribed RNA can function efficiently as a substrate for its self-encoded replicase.

As discussed in WO2011/005799, the self-replicating RNA can include (in addition to any 5′ cap structure) one or more nucleotides having a modified nucleobase. For instance, a self-replicating RNA can include one or more modified pyrimidine nucleobases, such as pseudouridine and/or 5 methylcytosine residues. In some embodiments, however, the RNA includes no modified nucleobases and may include no modified nucleotides i.e. all of the nucleotides in the RNA are standard A, C, G and U ribonucleotides (except for any 5′ cap structure, which may include a 7′ methylguanosine). In other embodiments, the RNA may include a 5′ cap comprising a 7′ methylguanosine, and the first 1, 2 or 3 5′ ribonucleotides may be methylated at the 2′ position of the ribose.

An RNA used with the invention for immunization purposes ideally includes only phosphodiester linkages between nucleosides, but in some embodiments it can contain phosphoramidate, phosphorothioate, and/or methylphosphonate linkages.

The invention includes embodiments where multiple species of RNAs are formulated with a lipid composition provided by the invention, such as 2, 3, 4, 5, 6, 7, 8, 9, 10, or more species of RNA, including different classes of RNA (such as mRNA, siRNA, self-replicating RNAs, and combinations thereof).

### Immunogens

In various embodiments, the saRNA molecules used in the invention can be used for immunization purposes, in some embodiments, encode a polypeptide immunogen. In these embodiments, after administration, the RNA is translated in vivo and the immunogen can elicit an immune response in the recipient. The immunogen may elicit an immune response against a pathogen (e.g. a bacterium, a virus, a fungus or a parasite) but, in some embodiments, it elicits an immune response against an allergen or a tumor antigen. The immune response may comprise an antibody response (usually including IgG) and/or a cell mediated immune response. The polypeptide immunogen will typically elicit an immune response which recognizes the corresponding pathogen (or allergen or tumor) polypeptide, but in some embodiments the polypeptide may act as a mimotope to elicit an immune response which recognizes a saccharide. The immunogen will typically be a surface polypeptide e.g. an adhesin, a hemagglutinin, an envelope glycoprotein, a spike glycoprotein, etc.

The RNA molecule can encode a single polypeptide immunogen or multiple polypeptides. Multiple immunogens can be presented as a single polypeptide immunogen (fusion polypeptide) or as separate polypeptides. If immunogens are expressed as separate polypeptides from an mRNA, then one or more of these may be provided with an upstream IRES or an additional viral promoter element. Alternatively, multiple immunogens may be expressed from a polyprotein that encodes individual immunogens fused to a short autocatalytic protease (e.g. foot-and-mouth disease virus 2A protein), or as inteins.

In certain embodiments, polypeptide immunogens (e.g., 1, 2, 3, 4, 5, 6, 7, 8, 9, 10, or more immunogens) may be used, either alone or together with a RNA molecule, such as a self-replicating RNA, encoding one or more immunogens (either the same or different as the polypeptide immunogens).

In some embodiments, the immunogen elicits an immune response against one of these bacteria:
Neisseria meningitidis: useful immunogens include, but are not limited to, membrane proteins such as adhesins, autotransporters, toxins, iron acquisition proteins, and factor H binding protein. A combination of three useful polypeptides is disclosed in Giuliani et al. (2006) Proc Natl Acad Sci USA 103 (29): 10834-9.
Streptococcus pneumoniae: useful polypeptide immunogens are disclosed in WO2009/016515. These include, but are not limited to, the RrgB pilus subunit, the beta-N-acetyl-hexosaminidase precursor (spr0057), spr0096, General stress protein GSP-781 (spr2021, SP2216), serine/threonine kinase StkP (SP1732), and pneumococcal surface adhesin PsaA.
Streptococcus pyogenes: useful immunogens include, but are not limited to, the polypeptides disclosed in WO02/34771 and WO2005/032582.
Moraxella catarrhalis.
Bordetella pertussis: Useful pertussis immunogens include, but are not limited to, pertussis toxin or toxoid (PT), filamentous haemagglutinin (FHA), pertactin, and agglutinogens 2 and 3.
Staphylococcus aureus: Useful immunogens include, but are not limited to, the polypeptides disclosed in WO2010/119343, such as a hemolysin, esxA, esxB, ferrichrome-binding protein (sta006) and/or the sta011 lipoprotein.
Clostridium tetani: the typical immunogen is tetanus toxoid.
Cornynebacterium diphtheriae: the typical immunogen is diphtheria toxoid.
Haemophilus influenzae: Useful immunogens include, but are not limited to, the polypeptides disclosed in WO2006/110413 and WO2005/111066.
Pseudomonas aeruginosa
Streptococcus agalactiae: useful immunogens include, but are not limited to, the polypeptides disclosed in WO02/34771.
Chlamydia trachomatis: Useful immunogens include, but are not limited to, PepA, LcrE, ArtJ, DnaK, CT398, OmpH-like, L7/L12, OmcA, AtoS, CT547, Eno, HtrA and MurG (e.g. as disclosed in WO2005/002619). LcrE (WO2006/138004) and HtrA (WO2009/109860) are two preferred immunogens.
Chlamydia pneumoniae: Useful immunogens include, but are not limited to, the polypeptides disclosed in WO02/02606.
Helicobacter pylori: Useful immunogens include, but are not limited to, CagA, VacA, NAP, and/or urease (WO03/018054).
Escherichia coli: Useful immunogens include, but are not limited to, immunogens derived from enterotoxigenic E. coli (ETEC), enteroaggregative E. coli (EAggEC), diffusely adhering E. coli (DAEC), enteropathogenic E. coli (EPEC), extraintestinal pathogenic E. coli (ExPEC) and/or enterohemorrhagic E. coli (EHEC). ExPEC strains include uropathogenic E. coli (UPEC) and meningitis/sepsis-associated E. coli (MNEC). Useful UPEC immunogens are disclosed in WO2006/091517 and WO2008/020330. Useful MNEC immunogens are disclosed in WO2006/089264. A useful immunogen for several E. coli types is AcfD (WO2009/104092).
Bacillus anthracis
Yersinia pestis: Useful immunogens include, but are not limited to, those disclosed in WO2007/049155 and WO2009/031043.
Staphylococcus epidermis
Clostridium perfringens or Clostridium botulinums
Legionella pneumophila
Coxiella burnetiid
Brucella, such as B. abortus, B. canis, B. melitensis, B. neotomae, B. ovis, B. suis, B. pinnipediae.
Francisella, such as F. novicida, F. philomiragia, F. tularensis
Neisseria gonorrhoeae
Treponema pallidum
Haemophilus ducreyi
Enterococcus faecalis or Enterococcus faecium
Staphylococcus saprophyticus
Yersinia enterocolitica
Mycobacterium tuberculosis
Rickettsia
Listeria monocytogenes
Vibrio cholerae
Salmonella typhi
Borrelia burgdorferi
Porphyromonas gingivalis
Klebsiella

In some embodiments the immunogen elicits an immune response against one of these viruses:
Orthomyxovirus: Useful immunogens can be from an influenza A, B or C virus, such as the hemagglutinin, neuraminidase or matrix M2 proteins. Where the immunogen is an influenza A virus hemagglutinin it may be from any subtype e.g. H1, H2, H3, H4, H5, H6, H7, H8, H9, H10, H11, H12, H13, H14, H15 or H16.
Paramyxoviridae viruses: immunogens include, but are not limited to, those derived from Pneumoviruses (e.g. respiratory syncytial virus, RSV), Rubulaviruses (e.g. mumps virus), Paramyxoviruses (e.g. parainfluenza virus), Metapneumoviruses and Morbilliviruses (e.g. measles virus).
Poxviridae: immunogens include, but are not limited to, those derived from Orthopoxvirus such as Variola vera, including but not limited to, Variola major and Variola minor.
Picornavirus: immunogens include, but are not limited to, those derived from Picornaviruses, such as Enteroviruses, Rhinoviruses, Heparnavirus, Cardioviruses and Aphthoviruses. In one embodiment, the enterovirus is a poliovirus e.g. a type 1, type 2 and/or type 3 poliovirus. In another embodiment, the enterovirus is an EV71 enterovirus. In another embodiment, the enterovirus is a coxsackie A or B virus.
Bunyavirus: immunogens include, but are not limited to, those derived from an Orthobunyavirus, such as California encephalitis virus, a Phlebovirus, such as Rift Valley Fever virus, or a Nairovirus, such as Crimean-Congo hemorrhagic fever virus.
Heparnavirus: immunogens include, but are not limited to, those derived from a Heparnavirus, such as hepatitis A virus (HAV).
Filovirus: immunogens include, but are not limited to, those derived from a filovirus, such as an Ebola virus (including a Zaire, Ivory Coast, Reston or Sudan ebolavirus) or a Marburg virus.
Togavirus: immunogens include, but are not limited to, those derived from a Togavirus, such as a Rubivirus, an Alphavirus, or an Arterivirus. This includes rubella virus.
Flavivirus: immunogens include, but are not limited to, those derived from a Flavivirus, such as Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus, Powassan encephalitis virus.
Pestivirus: immunogens include, but are not limited to, those derived from a Pestivirus, such as Bovine viral diarrhea (BVDV), Classical swine fever (CSFV) or Border disease (BDV).
Hepadnavirus: immunogens include, but are not limited to, those derived from a Hepadnavirus, such as Hepatitis B virus. A composition can include hepatitis B virus surface antigen (HBsAg).
Other hepatitis viruses: A composition can include an immunogen from a hepatitis C virus, delta hepatitis virus, hepatitis E virus, or hepatitis G virus.
Rhabdovirus: immunogens include, but are not limited to, those derived from a Rhabdovirus, such as a Lyssavirus (e.g. a Rabies virus) and Vesiculovirus (VSV).
Caliciviridae: immunogens include, but are not limited to, those derived from Calciviridae, such as Norwalk virus (Norovirus), and Norwalk-like Viruses, such as Hawaii Virus and Snow Mountain Virus.
Coronavirus: immunogens include, but are not limited to, those derived from COVID-19, a SARS coronavirus, avian infectious bronchitis (IBV), Mouse hepatitis virus (MHV), SARS, MERS, and Porcine transmissible gastroenteritis virus (TGEV). In addition, immunogens from bat and pangolin coronaviruses with pandemic potential can be used. The coronavirus immunogen may be a spike polypeptide or other virus proteins. Specific Coronavirus epitopes are comprehensively analyzed and described in Shrock et al, Science, Sep. 29, 2020 which is incorporated herein by reference.
Retrovirus: immunogens include, but are not limited to, those derived from an Oncovirus, a Lentivirus (e.g. HIV-1 or HIV-2) or a Spumavirus.
Reovirus: immunogens include, but are not limited to, those derived from an Orthoreovirus, a Rotavirus, an Orbivirus, or a Coltivirus.
Parvovirus: immunogens include, but are not limited to, those derived from Parvovirus B19.
Herpesvirus: immunogens include, but are not limited to, those derived from a human herpesvirus, such as, by way of example only, Herpes Simplex Viruses (HSV) (e.g. HSV types 1 and 2), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8).
Papovaviruses: immunogens include, but are not limited to, those derived from Papillomaviruses and Polyomaviruses. The (human) papillomavirus may be of serotype 1, 2, 4, 5, 6, 8, 11, 13, 16, 18, 31, 33, 35, 39, 41, 42, 47, 51, 57, 58, 63 or 65 e.g. from one or more of serotypes 6, 11, 16, and/or 18.
Adenovirus: immunogens include those derived from serotype 36 (Ad-36).

In some embodiments, the immunogen elicits an immune response against a virus which infects fish, such as: infectious salmon anemia virus (ISAV), salmon pancreatic disease virus (SPDV), infectious pancreatic necrosis virus (IPNV), channel catfish virus (CCV), fish lymphocystis disease virus (FLDV), infectious hematopoietic necrosis virus (IHNV), koi herpesvirus, salmon picorna-like virus (also known as picorna-like virus of atlantic salmon), landlocked salmon virus (LSV), atlantic salmon rotavirus (ASR), trout strawberry disease virus (TSD), coho salmon tumor virus (CSTV), or viral hemorrhagic septicemia virus (VHSV).

Fungal immunogens may be derived from Dermatophytres, including: Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var. album, var. discoides, var. ochraceum, Trichophyton violaceum, and/or Trichophyton faviforme; or from Aspergillus fumigatus, Aspergillus flavus, Aspergillus niger, Aspergillus nidulans, Aspergillus terreus, Aspergillus sydowii, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis and Enterocytozoon bieneusi; the less common are Brachiola spp, Microsporidium spp., Nosema spp., Pleistophora spp., Trachipleistophora spp., Vittaforma spp Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

In some embodiments the immunogen elicits an immune response against a parasite from the Plasmodium genus, such as P. falciparum, P. vivax, P. malariae or P. ovale. Thus, the invention may be used for immunizing against malaria. In some embodiments the immunogen elicits an immune response against a parasite from the Caligidae family, particularly those from the Lepeophtheirus and Caligus genera e.g. sea lice such as Lepeophtheirus salmonis or Caligus rogercresseyi.

In some embodiments the immunogen elicits an immune response against: pollen allergens (tree-, herb, weed-, and grass pollen allergens); insect or arachnid allergens (inhalant, saliva and venom allergens, e.g. mite allergens, cockroach and midges allergens, hymenopthera venom allergens); animal hair and dandruff allergens (from e.g. dog, cat, horse, rat, mouse, etc.); and food allergens (e.g. a gliadin). Important pollen allergens from trees, grasses and herbs are such originating from the taxonomic orders of Fagales, Oleales, Pinales and platanaceae including, but not limited to, birch (Betula), alder (Alnus), hazel (Corylus), hornbeam (Carpinus) and olive (Olea), cedar (Cryptomeria and Juniperus), plane tree (Platanus), the order of Poales including grasses of the genera Lolium, Phleum, Poa, Cynodon, Dactylis, Holcus, Phalaris, Secale, and Sorghum, the orders of Asterales and Urticales including herbs of the genera Ambrosia, Artemisia, and Parietaria. Other important inhalation allergens are those from house dust mites of the genus Dermatophagoides and Euroglyphus, storage mite e.g. Lepidoglyphys, Glycyphagus and Tyrophagus, those from cockroaches, midges and fleas e.g. Blatella, Periplaneta, Chironomus and Ctenocepphalides, and those from mammals such as cat, dog and horse, venom allergens including such originating from stinging or biting insects such as those from the taxonomic order of Hymenoptera including bees (Apidae), wasps (Vespidea), and ants (Formicoidae).

In some embodiments the immunogen is a tumor antigen selected from: (a) cancer-testis antigens such as NY-ESO-1, SSX2, SCP1 as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors; (b) mutated antigens, for example, p53 (associated with various solid tumors, e.g., colorectal, lung, head and neck cancer), p21/Ras (associated with, e.g., melanoma, pancreatic cancer and colorectal cancer), CDK4 (associated with, e.g., melanoma), MUM1 (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT; (c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), VVT 1 (associated with, e.g., various leukemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), mammaglobin, alpha-fetoprotein (associated with, e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer); (d) shared antigens, for example, melanoma-melanocyte differentiation antigens such as MART-1/Melan A, gp100, MC1R, melanocyte-stimulating hormone receptor, tyrosinase, tyrosinase related protein-1/TRP1 and tyrosinase related protein-2/TRP2 (associated with, e.g., melanoma); (e) prostate associated antigens such as PAP, PSA, PSMA, PSH-P1, PSM-P1, PSM-P2, associated with e.g., prostate cancer; (f) immunoglobulin idiotypes (associated with myeloma and B cell lymphomas, for example). In certain embodiments, tumor immunogens include, but are not limited to, p15, Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, p185erbB2, p180erbB-3, c-met, mn-23H1, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29BCAA), CA 195, CA 242, CA-50, CAM43, CD68KP1, CO-029, FGF-5, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, TPS, and the like.

### Vaccine Compositions

A pharmaceutical composition of the invention, particularly one useful for immunization, may include one or more small molecule immunopotentiators. For example, the composition may include a TLR2 agonist (e.g. Pam3CSK4), a TLR4 agonist (e.g. an aminoalkyl glucosaminide phosphate, such as E6020), a TLR7 agonist (e.g. imiquimod), a TLR8 agonist (e.g. resiquimod) and/or a TLR9 agonist (e.g. IC31). Any such agonist ideally has a molecular weight of <2000 Da. Such agonist(s) can, in some embodiments, be encapsulated with the RNA inside LNPs, or encapsulated or complexed with LNPs, but in other embodiments they are unencapsulated or not complexed. In some embodiments, adjuvants are for example: montanide ISA-51 (Seppic Inc., Fairfield, N.J., United States of America); QS-21 (Aquila Biopharmaceuticals. Inc., Framingham, Mass., United States of America); Arlacel A; oeleic acid; tetanus helper peptides (such as but not limited to QYIKANSKFIGITEL (SEQ ID NO: 2376) and/or AQYIKANSKFIGITEL (SEQ ID NO: 2377); GM-CSF; cyclophosamide; bacillus Calmette-Guerin (BCG); Corynbacterium parvum; levamisole, azimezone; isoprinisone; dinitrochlorobenezene (DNCB); keyhole limpet hemocyanin (KLH); Freunds adjuvant (complete and incomplete); mineral gels; aluminum hydroxide (Alum); lysolecithin; pluronic polyols; polyanions; peptides; oil emulsions; nucleic acids (such as but not limited to soluble-stranded RNAs; dsRNA) dinitrophenol; diphtheria toxin (DT); toll-like receptor (TLR; such as but not limited to TLR3, TLR4, TLR7, TLR8, and/or TLR9) agonists (including but not limited to endotoxins such as lipopolysaccharide (LPS); monophosphoryl lipid A (MPL); and/or polyinosinic-polycytidylic acid (poly-ICLC/HILTONOL.RTM.; Oncovir, Inc., Washington, D.C., United States of America); IMO-2055; glucopyranosyl lipid A (GLA); QS-21 (a saponin extracted from the bark of the Quillaja saponaria tree, also known as the soap bark tree or Soapbark); resiquimod (a TLR7/8 agonist); CDX-1401 (a fusion protein consisting of a fully human monoclonal antibody with specificity for the dendritic cell receptor DEC-205 linked to the NY-ESO-1 tumor antigen); Juvaris' Cationic Lipid-DNA Complex; Vaxfectin; and combinations thereof. In one embodiment, adjuvants using heterogeneous monophosphoryl Lipid A (MPL) derived from Salmonella minnesota R595 are used to induce Th-1 type immune responses to heterologous proteins in animal and human vaccines. Exemplary monophosphoryl Lipid A type adjuvants are shown below:

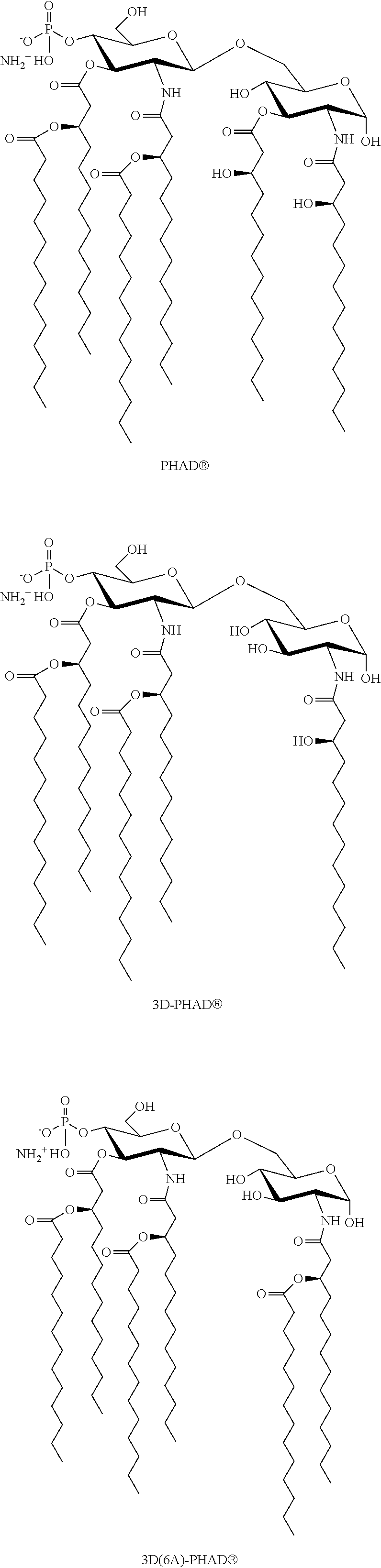

Pharmaceutical compositions of the invention may have an osmolality of about or at least 100, 150, 175, 200, 250, 275, 300, 325, 350, 375, 400, 425, 450, 475, 500 mOsm/kg. In some embodiments, osmolality between about 200 mOsm/kg and 400 mOsm/kg, e.g. between about 240-360 mOsm/kg, or between about 290-310 mOsm/kg.

Pharmaceutical compositions of the invention may include one or more preservatives, such as thiomersal or 2 phenoxyethanol. Mercury-free compositions can be made, and preservative-free vaccines can be prepared. Compositions comprise an immunologically effective amount of lipid compositions described herein (e.g., liposomes and LNPs), as well as any other components, as needed. Immunologically effective amount refers to the amount administered to an individual, either in a single dose or as part of a series, which is effective for treatment (e.g., prophylactic immune response against a pathogen). This amount varies depending upon the health and physical condition of the individual to be treated, age, the taxonomic group of individual to be treated (e.g. non-human primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials. The compositions of the invention will generally be expressed in terms of the amount of RNA per dose.

The invention also provides a delivery device (e.g. syringe, nebulizer, sprayer, inhaler, dermal patch, etc.) containing a pharmaceutical composition of the invention. This device can be used to administer the composition to a vertebrate subject.

### Methods of Treatment and Medical Uses

LNP-formulated saRNA and pharmaceutical compositions described herein are for in vivo use for inducing an immune response against an immunogen of interest.

The invention provides a method for inducing an immune response in a vertebrate comprising administering an effective amount of the liposome-formulated or LNP-formulated saRNA, or pharmaceutical composition, as described herein. The immune response is preferably protective and preferably involves antibodies and/or cell-mediated immunity. The compositions may be used for both priming and boosting purposes. Alternatively, a prime-boost immunization schedule can be a mix of RNA and the corresponding polypeptide antigen (e.g., RNA prime, protein boost).

The invention also provides a liposome, LNP, or pharmaceutical composition for use in inducing an immune response in a vertebrate. The invention also provides the use of a liposome, LNP, or pharmaceutical composition in the manufacture of a medicament for inducing an immune response in a vertebrate.

By inducing an immune response in the vertebrate by these uses and methods, the vertebrate can be protected against various diseases and/or infections e.g. against bacterial and/or viral diseases as discussed above. The liposomes, LNPs, and compositions are immunogenic, and are more preferably vaccine compositions. Vaccines according to the invention may either be prophylactic (i.e. to prevent infection) or therapeutic (i.e. to treat infection) but will typically be prophylactic.

The vertebrate is preferably a mammal, such as a human or a large veterinary mammal (e.g. horses, cattle, deer, goats, pigs). As used herein “large mammal” refers to mammals having a typical or average adult weight of at least 5 kg, preferably at least 7 kg. Such large mammals can include, for example, humans, non-human primates, dogs, pigs, cattle, deer, goats, and is meant to exclude small mammals, such as mice, rats, guinea pigs, and other rodents.

Where the vaccine is for prophylactic use, the human is preferably a child (e.g. a toddler or infant) or a teenager; where the vaccine is for therapeutic use, the human is preferably a teenager or an adult. A vaccine intended for children may also be administered to adults e.g. to assess safety, dosage, immunogenicity, etc.

Vaccines prepared according to the invention may be used to treat both children and adults. Thus, a human patient may be less than 1 year old, less than 5 years old, 1-5 years old, 5-15 years old, 15-55 years old, or at least 55 years old. Preferred patients for receiving the vaccines are the elderly (e.g., ≥50 years old, ≥60 years old, and preferably ≥65 years), the young (e.g. <5 years old), hospitalized patients, healthcare workers, armed service and military personnel, pregnant women, the chronically ill, or immunodeficient patients. The vaccines are not suitable solely for these groups, however, and may be used more generally in a population. Compositions of the invention will generally be administered directly to a patient. Direct delivery may be accomplished by parenteral injection (e.g. subcutaneously, intraperitoneally, intravenously, intramuscularly, intradermally, or to the interstitial space of a tissue; intraglossal injection is not typically used for immunization purposes. Alternative delivery routes include rectal, oral (e.g. tablet, spray), buccal, sublingual, vaginal, topical, transdermal or transcutaneous, intranasal, ocular, aural, pulmonary or other mucosal administration. Intradermal and intramuscular administration are two preferred routes. Injection may be via a needle (e.g. a hypodermic needle), but needle-free injection may alternatively be used. A typical intramuscular dose is 0.5 ml.

The invention may be used to induce systemic and/or mucosal immunity, preferably to elicit an enhanced systemic and/or mucosal immunity.

Dosage can be by a single dose schedule or a multiple dose schedule. Multiple doses may be used in a primary immunization schedule and/or in a booster immunization schedule. In a multiple dose schedule the various doses may be given by the same or different routes e.g. a parenteral prime and mucosal boost, a mucosal prime and parenteral boost, etc. Multiple doses will typically be administered at least 1 week apart (e.g. about 2 weeks, about 3 weeks, about 4 weeks, about 6 weeks, about 8 weeks, about 10 weeks, about 12 weeks, about 16 weeks, etc.). In one embodiment, multiple doses may be administered approximately 6 weeks, 10 weeks and 14 weeks after birth, e.g. at an age of 6 weeks, 10 weeks and 14 weeks, as often used in the World Health Organization's Expanded Program on Immunization (“EPI”). In an alternative embodiment, two primary doses are administered about two months apart, e.g. about 7, 8 or 9 weeks apart, followed by one or more booster doses about 6 months to 1 year after the second primary dose, e.g. about 6, 8, 10 or 12 months after the second primary dose. In a further embodiment, three primary doses are administered about two months apart, e.g. about 7, 8 or 9 weeks apart, followed by one or more booster doses about 6 months to 1 year after the third primary dose, e.g. about 6, 8, 10, or 12 months after the third primary dose.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

Summary: LNPs are formulated using total lipid concentration of 10 to 25 mM comprised of, for example, KC2/DSPC/Cholesterol/PEG-DMG (50:10:38.5:1.5 mol %). Each one of the lipids were solubilized in ethanol until a clear solution was observed. The four lipids are combined to obtain 50 mM total lipid concentration (25/5/19.25/0.75 mM respectively), then serial dilutions are done to reach 10 to 25 mM.

A. Firefly Luciferase Assay for saRNA Delivery Efficiency. After 24 hours of transfection, transfected cells are conditioned to room temperature for 30 minutes prior to the Firefly Luciferase Assay. Quantilum Recombinant Luciferase standard curve was prepared in 10% EMEM in 5-fold serial dilutions. 50 ul of each standard point from the range of 3.9×10⁻⁵ mg/ml to 4.88×10⁻³ mg/ml are included in the microplate as a positive enzyme activity control to maintain a linearity of 107 RLU/mg/ml. The ONE-Glo substrate, previously conditioned to room temperature for at least 4 hours, is added to each untransfected, transfected and Quantilum wells in a ratio 1:1. Assay plates are incubated for 3 minutes in darkness and immediately introduced into the Cytation 5 Cell Imaging Multi-Mode Reader (Biotek) to read luminescence.

B: Ribogreen Assay for mRNA Encapsulation Efficiency. 1×TE Buffer and Triton Buffer (2% v/v in 1×TE Buffer) are added in duplicates into a black microplate per LNP. LNPs are diluted to 4 ng/ul in 1×DPBS pH 7.4 and are added to each TE/Triton well in a ratio 1:1. Two standard curves are included in the Ribogreen Assay, one containing mRNA and 1×TE Buffer and other containing mRNA and Triton Buffer. Each one of these standard curves are used to calculate the mRNA concentration in each TE Buffer or Triton Buffer. This approach using two standard curves is more accurate for calculating the encapsulation efficiency and mRNA concentrations, in comparison to a single standard curve. Standards are included in the microplate after diluted LNPs are added to the plate. Microplates are incubated at 37° C. for 10 minutes to extract LNPs with Triton. Ribogreen reagent is diluted 1:100 in 1×TE Buffer and added to each well in a ratio 1:1. Microplates are immediately introduced into the Cytation 5 Cell Imaging Multi-Mode Reader (Biotek) to read Fluorescence (Ex485/Em528).

C: Dynamic Light Scattering for LNP Size (red dots are PDI right y axis). LNPs are diluted to 6.25 ng/ul in 1×DPBS pH 7.4 and are transferred into a quartz cuvette (ZEN2112) to measure size by Dynamic Light Scattering (DLS) in the Zetasizer Nano ZS (Malvern Panalytical) using particle RI of 1.45 and Absorption of 0.001 in 1×PBS at 25° C. with viscosity of 1.02 cP and RI of 1.335. Measurements are made using a 173° Backscatter angle of detection previously equilibrated to 25° C. for 30 seconds in duplicates, each with 5 runs and 10 second run duration, without delay between measurements. Each measurement has a fixed position of 4.65 in the quartz cuvette with an automatic attenuation selection. Data is analyzed using a General-Purpose model with normal resolution.

### Example 2: Swine Studies

Swine Study 1: saRNA-LNPs prepared by Direct Dilution are effective in vivo. Four (4) pigs immunized with Swine Influenza Virus H1N1 (SIV) Strain [A/Swine/IA/A01778877/2016 (H1N1) saRNA vaccines targeting H1 hemagglutinin utilizing a dialyzed preparation of LNP vaccines with cryobuffer (Dialysis) or Direct Dilution utilizing proprietary ionizable lipids. Included in this study is a placebo control for comparison (n=2). Pigs were immunized at approximately 3 weeks of age and administered a boost immunization 28 days later. Data represents H1 specific ELISA data at study day 56, 28 days post boost immunization as well as hemagglutination inhibition (HI) data targeting H1 influenza virus.

TABLE 5

| | ELISA | |
|---|---|---|
| Method | OD450 | Hi Titer |
| Placebo | 0.16 +/− 0.04 | 0 +/− 0 |
| Dialysis | 0.37 +/− 0.13 | 0 +/− 0 |
| Direct Dilution | 0.50 +/− 0.05 | 15 +/− 9 |

Values are the mean +/− SEM

Swine study 2: Ten (10) commercial pigs immunized with Swine Influenza Virus H1N1 (SIV) Strain [A/Swine/IA/A01778877/2016 (H1N1) saRNA vaccines targeting H1 hemagglutinin utilizing a direct dilution with cryobuffer at a total dose of 1 μg. Included in this study is a placebo control for comparison (n=2). Pigs were immunized at approximately 3 weeks of age and administered a boost immunization 28 days later. Data represents H1 specific ELISA data at study day 56, 28 days post boost immunization as well as hemagglutination inhibition (HI) data targeting H1 influenza virus.

TABLE 6

| | ELISA | |
|---|---|---|
| Method | OD450 | Hi Titer |
| Placebo | 0.12 +/− 0.04 | 0 +/− 0 |
| Direct Dilution | 0.44 +/− 0.06 | 21 +/− 8 |

Values are the mean +/− SEM

Table 7. Swine Study 3: Groups of 3-4 commercial pigs immunized with Swine Influenza Virus H1N1 (SIV) Strain [A/Swine/IA/A01778877/2016 (H1N1) saRNA vaccines targeting H1 hemagglutinin utilizing a direct dilution with cryobuffer at a total dose of 0.5 μg. Included in this study is a placebo control for comparison (n=3). Pigs were immunized at approximately 3 weeks of age and administered a boost immunization 28 days later. At 51 days post initial immunization pigs were dosed with a virulent challenge of SIV H1N1 intratracheally. Five (5) days post challenge, pigs were humanely euthanized, and lungs scored by a pathologist for lesion consolidation consistent with SIV. Data represents lung lesion consolidation scoring at study day 56. ELISA data is assessed prior to challenge; HI titer data was assessed post-challenge with virulent virus.

TABLE 7

| | ELISA | | Lung Lesion |
|---|---|---|---|
| Method | OD450 | Hi Titer | Consolidation |
| Placebo | 0.15 +/− 0.04 | 0 +/− 0 | 12.7 +/− 5.5 |
| Dialysis | 0.18 +/− 0.05 | 105 +/− 73 | 7.3 +/− 3.3 |
| Direct Dilution | 0.35 +/− 0.10 | 200 +/− 76 | 8.0 +/− 4.1 |

Values are the mean +/− SEM

Table 8. Poultry Study 1: Groups of 24 commercial broilers were immunized with Highly Pathogenic Avian Influenza (HPAI) H5N1 Strain [A/American wigeon/South Carolina/AH0195145/2021 (H5N1)] saRNA vaccines targeting H5 hemagglutinin utilizing a direct dilution with cryobuffer at a dose of 1 μg. Included in this study is a placebo control for comparison (n=24). Eight (8) unique lipid nanoparticle formulations utilizing different proprietary ionizable lipids were utilized to assess serological conversion and responses post immunization. Birds were immunized at approximately 1 day of age and administered a boost immunization 7 days later. At 28 days post initial immunization blood samples were obtained from the birds and assessed for specific antibody response against H5 hemagglutinin. Data represents H5 specific ELISA data at study day 28d.

TABLE 8

| | ELISA | |
|---|---|---|
| Method | OD450 | HI Titer |
| Placebo | 0.15 +/− 0.02 | 0 +/− 0 |
| LNP1 | 1.01 +/− 0.12 | 3.14 +/− 0.9 |
| LNP2 | 0.23 +/− 0.03 | 0 +/− 0 |
| LNP3 | 0.43 +/− 0.07 | 0.69 +/− 0.40 |
| LNP4 | 0.40 +/− 0.06 | 0.17 +/− 0.17 |
| LNP5 | 0.67 +/− 0.12 | 1.18 +/− 0.60 |
| LNP6 | 0.33 +/− 0.05 | 0.76 +/− 0.44 |
| LNP7 | 1.29 +/− 0.12 | 2.70 +/− 0.57 |
| LNP8 | 0.66 +/− 0.11 | 2.09 +/− 1.42 |

Values are the mean +/− SEM

The collective data from swine and poultry studies demonstrate the applicability of the direct dilution methodology for vaccine preparation. In swine, direct dilution of saRNA vaccines with cryobuffer consistently elicited H1-specific humoral responses, as measured by ELISA and hemagglutination inhibition (HI) assays, across multiple studies using different dose levels. Notably, pigs receiving directly diluted vaccine formulations showed measurable immunogenicity and, in the case of the challenge study, a dose-responsive reduction in lung lesion scores compared to placebo controls. Similarly, in poultry, direct dilution with cryobuffer enabled effective immunization with multiple lipid nanoparticle formulations, resulting in detectable H5-specific antibody responses. These findings collectively support the utility and performance of the direct dilution approach for preparing effective RNA-based vaccines across species.

For all patents, applications, or other reference cited herein, such as non-patent literature and reference sequence information, it should be understood that they are incorporated by reference in their entirety for all purposes as well as for the proposition that is recited. Where any conflict exists between a document incorporated by reference and the present application, this application will control. All information associated with reference gene sequences disclosed in this application, such as GeneIDs or accession numbers (typically referencing NCBI accession numbers), including, for example, genomic loci, genomic sequences, functional annotations, allelic variants, and reference mRNA (including, e.g., exon boundaries or response elements) and protein sequences (such as conserved domain structures), as well as chemical references (e.g., PubChem compound, PubChem substance, or PubChem Bioassay entries, including the annotations therein, such as structures and assays, et cetera), are hereby incorporated by reference in their entirety.

Headings used in this application are for convenience only and do not affect the interpretation of this application.

Preferred features of each of the aspects provided by the invention are applicable to all of the other aspects of the invention mutatis mutandis and, without limitation, are exemplified by the dependent claims and also encompass combinations and permutations of individual features (e.g., elements, including numerical ranges and exemplary embodiments) of particular embodiments and aspects of the invention, including the working examples. For example, particular experimental parameters exemplified in the working examples can be adapted for use in the claimed invention piecemeal without departing from the invention. For example, for materials that are disclosed, while specific reference of each of the various individual and collective combinations and permutations of these compounds may not be explicitly disclosed, each is specifically contemplated and described herein. Thus, if a class of elements A, B, and C are disclosed as well as a class of elements D, E, and F and an example of a combination of elements A-D is disclosed, then, even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-groups of A-E, B-F, and C-E are specifically contemplated and should be considered disclosed from disclosure of A, B, and C; D, E, and F; and the example combination A-D. This concept applies to all aspects of this application, including elements of a composition of matter and steps of method of making or using the compositions.

The forgoing aspects of the invention, as recognized by the person having ordinary skill in the art following the teachings of the specification, can be claimed in any combination or permutation to the extent that they are novel and non-obvious over the prior art-thus, to the extent an element is described in one or more references known to the person having ordinary skill in the art, they may be excluded from the claimed invention by, inter alia, a negative proviso or disclaimer of the feature or combination of features.

What is claimed is:

1. A dialysis-free method for making a lipid nanoparticle (“LNP”) comprising a nucleic acid comprising:
a. providing a nucleic acid solution comprising at least one nucleic acid;
b. providing a lipid solution in an organic solvent comprising at least one lipid;
c. combining a portion of the nucleic acid solution and a portion of the lipid solution to create a mixing solution; and
d. adjusting the pH in the mixing solution to physiological pH to obtain a pH-adjusted mixing solution; and
e. diluting the pH-adjusted mixing solution using a buffer to obtain an ethanol concentration of below about 350 mM to obtain a final nucleic acid encapsulated LNP solution, wherein the final nucleic acid encapsulated LNP solution is obtained without using a dialysis step.

2. The method of claim 1, wherein the portion of the nucleic acid solution and the portion of the lipid solution are combined in step (1c) to obtain a volume ratio (v/v) selected from the group consisting of 1:1, 2:1, 3:1, 4:1, 5:1, 6:1 and 7:1.

3. The method of claim 1, wherein the LNPs have an average diameter in the range of about 40 to about 150 nanometers.

4. The method of claim 1, wherein the LNPs have an average diameter in the range of about 50 to about 100 nanometers.

5. The method of claim 1 wherein the LNPs have a nucleic acid encapsulation efficiency of about 40 to about 100%

6. The method of 1, wherein the at least one nucleic acid is DNA or RNA.

7. The method of claim 1, wherein the at least one nucleic acid is mRNA.

8. The method of claim 1, wherein the at least one nucleic acid is saRNA.

9. The method of claim 1, wherein the at least one nucleic acid is saRNA encoding at least one open reading frame.

10. The method of claim 1, wherein the at least one nucleic acid is saRNA encoding at least one open reading frame encoding an immunogen.

11. The method of claim 1, wherein the organic solvent is selected from the group consisting of ethanol and acetone.

12. The method of claim 1, wherein the organic solvent is ethanol.

13. The method of claim 1, wherein the buffer in step (1e) used to dilute the pH-adjusted mixing solution is a cryoprotectant buffer.

14. The method of claim 13, wherein the diluting to obtain nucleic acid encapsulated LNPs to an ethanol concentration of below about 350 mM is achieved using a cryoprotectant buffer.

15. The method of claim 14, wherein the ethanol concentration is below about 100 mM.

16. The method of claim 14, wherein the ethanol concentration is below 50 mM.
